(19) United States
(12) Patent Application Publication
(10) Pub. No.: US 2024/0002447 A1
(43) Pub. Date: Jan. 4, 2024

(54) MODIFIED HUMAN PAPILLOMAVIRUS TYPE 52 L1 PROTEIN AND USE THEREOF

(71) Applicant: Institute of Basic Medical Sciences, Chinese Academy of Medical Sciences, Beijing (CN)

(72) Inventors: Xuemei Xu, Beijing (CN); Mingrao Ma, Beijing (CN); Yaru Hao, Beijing (CN); Ting Zhang, Beijing (CN); Zhirong Wang, Beijing (CN)

(21) Appl. No.: 18/254,576

(22) PCT Filed: Sep. 26, 2021
(86) PCT No.: PCT/CN2021/120518

§ 371 Date: May 25, 2023

(30) Foreign Application Priority Data
Nov. 26, 2020 (CN) 202011351390.9

(51) Int. Cl. C07K 14/005; A61K 39/12

(52) U.S. Cl. CPC C07K 14/005; A61K 39/12; C12N 2710/20022; C12N 2710/20034; C12N 2710/20023; C12N 2710/20071; A61K 2039/5258

(57) ABSTRACT

The present application relates to a modified human papillomavirus (HPV) type 52 L1 protein and a use thereof. Specifically, the present application relates to a HPV type 52 L1 protein, a nucleotide encoded thereby, a carrier comprising the nucleotide, a cell comprising the carrier, a pentamer or virus-like particle consisting of the HPV-52 L1 protein, a vaccine comprising the pentamer or virus-like particle and a vaccine adjuvant, and a use thereof in the prevention of HPV infections and HPV infection-related diseases.

### CROSS-REFERENCE TO RELATED APPLICATIONS

This application is a U.S. National Phase of International PCT Application No. PCT/CN2021/120518 filed on Sep. 26, 2021, which claims priority to Chinese Patent Application Serial No. 202011351390.9 filed on Nov. 26, 2020, the contents of each application are incorporated herein by reference in their entireties.

### SEQUENCE LISTING

This application incorporates by reference the material in the ASCII text file titled English_Translation_of_Sequence_Listing.txt, which was created on May 16, 2023 and is 170 KB.

### FIELD OF THE INVENTION

The present application relates to the field of biotechnology. Specifically, the present application relates to a novel human papillomavirus protein, and a pentamer or a virus-like particle formed thereby, as well as use of the human papillomavirus protein, the pentamer or the human papillomavirus-like particle in the preparation of a vaccine for use in the prevention of papillomavirus infection and infection-induced diseases.

### BACKGROUND OF THE INVENTION

Human papillomavirus (HPV) is a class of non-enveloped small DNA viruses that infect epithelial tissue. More than 200 types of HPV have been identified, which can be classified into mucosal types and skin types according to the different sites of infection. Mucosal type HPVs mainly infect the urogenital, perianal and oropharyngeal mucous membrane and skin. HPVs can also be classified into carcinogenic types that have transforming activity and low-risk types that induce benign hyperplasia. There are more than 20 carcinogenic types, including 12 common high-risk types (HPV16, -18, -31, -33, -35, -39, -45, -51, -52, -56, -58, -59) and more than 10 relatively rare possible/suspected high-risk types (HPV26, -30, -34, -53, -66, -67, -68, -69, -70, -73, -82), the persistent infection of which can induce about 100% of cervical cancer, 88% of anal cancer, 70% of vaginal cancer, 50% of penile cancer, 43% of vulva cancer and 72% of head and neck cancer. Among these cancers, cervical cancer has the third highest incidence of women's malignant tumors in the world, and the second highest incidence of women's malignant tumors in the population of women aged 15-44, second only to breast cancer. There are about 570,000 cases and 311,000 deaths per year, of which more than 80% occur in underdeveloped countries and regions.

HPV52 is a relatively common dominant prevalent virus strain worldwide, with a detection rate of 3.5% in cervical cancer tissue, ranking sixth. It is also worth noting that in normal cervical tissues or cervical tissues with low-grade lesions in China, the detection rate of HPV52 reaches 2.8% and 16%, both ranking first. In addition, the detection rate of HPV52 in cervical cancer tissues in southern China ranks third after HPV16 and HPV18.

The major capsid protein L1 of HPV self-assembles to form virus-like particles (VLPs), which mainly induce type-specific neutralizing antibodies and protective activity. At present, the four HPV prophylactic vaccines on the market are all L1VLP combination vaccines, namely the HPV16/-18 L1VLP bivalent vaccine (Cervarix) produced by insect expression systems, the HPV161-181-6/-11 L1VLP tetravalent vaccine (Gardasil) and the HPV16/-18/-31/-33/-45/-52/-58/-6/-11 L1VLP nine-valent vaccine (Gardasil-9) produced by yeast expression systems, and the HPV16/-18 bivalent vaccine (Cecolin) produced by prokaryotic expression systems. Yet, currently, only Gardasil-9 comprises HPV52 L1VLP.

At present, the relatively commonly used VLP expression systems include prokaryotic expression systems, yeast expression systems and insect expression systems. It was found by comparing the clinical data of the marketed Cervarix and Gardasil that the content of HPV16 L1VLP in Cervarix (20 μg) was only half of that in Gardasil (40 μg), and the content of HPV18 L1VLP in Cervarix was the same as that of Gardasil (both 20 μg). But, the type-specific neutralizing antibody titers against HPV16 and HPV18, cross-neutralization activity, memory B cell number and CD4+ T cell response level induced by Cervarix were all higher than those induced by Gardasil, indicating that the immune activity of Cervarix was stronger than that of Gardasil. Furthermore, insect cell expression systems have many advantages. Compared with prokaryotic expression systems, insect cell expression systems have relatively close genetic distance to the natural host cell of the virus (both are eukaryotic multicellular organisms), do not contain endotoxins, and proteins are mostly expressed in soluble forms therein without the trouble of inclusion bodies. Compared with yeast expression systems, insect cells are easy to lyse and the purification process is relatively simple, while disruption of yeast cell wall requires high-pressure homogenization, and the presence of more host proteins causes relatively more difficulties in purification. Therefore, insect expression systems are more advantageous for developing vaccines. However, the fermentation cost of insect expression systems is relatively high, so it is especially important to increase the expression level and yield of L1VLP to reduce the cost of vaccine production.

It was found that optimizing an antigen gene according to the biased codons of the host cell could increase its expression level. For example, optimizing HPV11 L1 gene with mammalian cell biased codons increased its expression level in human embryonic kidney cells (293T) by at least 100 folds. The expression level and VLP yield of HPV16 L1 variant strains in insect and yeast expression systems were analyzed and compared. It was found that when high-frequency mutation sites were mutated into dominant amino acids, the L1 expression level and VLP yield would increase. But when high-frequency mutations sites in combination with other sites were mutated, the effect on the L1 expression level was uncertain. In insect expression systems, BPV1 L1 was modified by C-terminus truncation, and it was found that the assembly efficiency of truncated BPV L1 increased by 3 folds. At present, the effect of C-terminus truncation on protein expression amount has not been reported. In prokaryotic expression systems, L1 of HPV16, -18, -31, -33, -45, -52, -58, -6, and -11 types was modified by N-terminus truncation, and it was found that the number of amino acids truncated at N-terminus that could upregulate the L1 expression level varied from type to type and was irregular.

It has been found in the present application that optimal modification of the N-terminus, C-terminus and high-frequency mutation sites of L1 can significantly increase the expression level and yield of 52L1VLP, and the obtained HPV52 L1VLP can induce high titers of type-specific neutralizing antibodies.

### SUMMARY OF THE INVENTION

Some embodiments of the present application provide a novel optimally modified HPV52 L1 protein, a pentamer or a virus-like particle composed thereof, and a vaccine containing the pentamer or virus-like particle, and study use of the vaccine in the prevention of HPV infection and infection-related diseases.

The inventor has unexpectedly found that the expression amount of HPV52 L1 protein in insect cell expression systems can be increased by appropriate amino acid substitution of high-frequency mutation sites of HPV52 L1 protein and partial deletion or amino acid substitution of its N-terminus and/or C-terminus. The optimally modified protein can be assembled into VLP and can induce a protective immune response against HPV52.

Thus, according to some embodiments of the present application, the present application relates to an optimally modified HPV52 L1 protein comprising a modification selected from the group consisting of the following or a combination thereof, compared with wild-type HPV52 L1 protein (for example, the amino acid sequence corresponding to the sequence AEI61557.1 in NCBI database):
mutating the amino acid at position 447 from aspartate to glutamate;
deleting 1 to 20 successive or nonsuccessive amino acids at the N-terminus;
deleting 1 to 25 successive or nonsuccessive amino acids at the C-terminus;
substituting one or more amino acids at positions 1 to 20 at the N-terminus;
substituting one or more amino acids at positions 1 to 25 at the C-terminus.

Specifically, according to some embodiments of the present application, provided is an optimally modified HPV52 L1 protein, wherein the modified HPV52 L1 protein has any feature selected from the group consisting of the following or a combination thereof, compared with wild-type HPV52 L1 protein:
mutating the amino acid at position 447 from aspartate (D) to glutamate (E);
deleting 1-20 successive/nonsuccessive amino acids at the N-terminus (for example, 1, 2, 3, 4, 5, 6, 7, 8, 9, 10, 11, 12, 13, 14, 15, 16, 17, 18, 19, 20 amino acids);
deleting 13 amino acids at the N-terminus and substituting with serine (S), serine-glutamate (SE), serine-glutamate-arginine (SER), or proline-serine-glutamate-alanine-threonine (PSEAT);
deleting 1-25 successive/nonsuccessive amino acids at the C-terminus (for example, 1, 2, 3, 4, 5, 6, 7, 8, 9, 10, 11, 12, 13, 14, 15, 16, 17, 18, 19, 20, 21, 22, 23, 24, 25 amino acids);
substituting one or more basic amino acids at positions 1-23 at the C-terminus with polar uncharged amino acids, non-polar amino acids and/or acidic amino acids.

In particular embodiments, the basic amino acid is arginine (R) and/or lysine (K).

In particular embodiments, the polar uncharged amino acid is glycine (G), serine (S) and/or threonine (T).

In particular embodiments, the non-polar amino acid is alanine (A) and/or valine (V).

In particular embodiments, the acidic amino acid is aspartate (D) and/or glutamate (E).

In particular embodiments, the optimally modified HPV52 L1 protein according to the present application is modified on the basis of the sequence as shown in SEQ ID No. 1 (the amino acid sequence corresponding to the sequence AEI61557.1 in NCBI database).

In particular embodiments, the modified HPV52 L1 protein is selected from the group consisting of 52L1D447EΔC19, 52L1ΔN2, 52L1ΔN4, 52L1ΔN5, 52L1ΔN8, 52L1ΔN10, 52L1ΔN13, 52L1ΔN15, 52L1ΔN18, 52L1ΔN20, 52L1CS1, 52L1CS2, 52L1CS3, 52L1CS4, 52L1CS5, 52L1CS6, 52L1CS7, 52L1CS8, 52L1CS9, 52L1ΔN13CS1, 52L1ΔN13CS2, 52L1ΔN13CS3, 52L1NS1ΔC19, 52L1NS1ΔC25, 52L1NS2ΔC19, 52L1NS3ΔC19, 52L1NS4ΔC19 and 52L1ΔN144C25, the amino acid sequences of which are as shown in SEQ ID No. 2 to SEQ ID No. 29.

The wild-type HPV52 L1 protein can also be from, but not limited to, L1 proteins from HPV52 variant strains, such as ABU55797.1, AEI61589.1, AIF71344.1, APQ44868.1, AEI61581.1, AIF71350.1, CAD1814034.1, etc. in NCBI database, and C-terminus modified L1 proteins corresponding to the variant strains are modified in the same way as those for the above-mentioned modified HPV52 L1 protein, such as evaluated by sequence comparison.

According to some embodiments of the present application, provided is a polynucleotide encoding the optimally modified HPV52 L1 protein of the present application. Preferably, the polynucleotide is optimized using codons of commonly used expression systems, such as E. coli expression systems, yeast expression systems, insect cell expression systems, etc. Preferably, the polynucleotide is optimized using insect cell codons.

According to some embodiments of the present application, provided is a vector containing the above-mentioned polynucleotide. Preferably, the vector is selected from the group consisting of plasmid, recombinant Bacmid and recombinant baculovirus.

According to some embodiments of the present application, provided is a cell comprising the above-mentioned vector. Preferably, the cell is an E. coli cell, a yeast cell or an insect cell, and particularly preferably, the cell is an insect cell.

According to some embodiments of the present application, provided is a HPV52 L1 multimer (e.g., pentamer) or a virus-like particle, the multimer or virus-like particle contains the above-mentioned modified HPV52 L1 protein or is formed thereby.

According to some embodiments of the present application, provided is a vaccine for the prevention of HPV infection or HPV infection-related diseases comprising the above-mentioned multimer or virus-like particle, wherein the content of the multimer or virus-like particle is an effective amount that can induce a protective immune response. Preferably, the vaccine can also comprise at least one selected from other mucosa-tropic and/or skin-tropic HPV pentamer or virus-like particle, the content of which is an effective amount that can induce a protective immune response, respectively. The above-mentioned vaccine usually also comprises an excipient or carrier for vaccines.

In particular embodiments, the vaccine contains the above-mentioned HPV52 L1 multimer (e.g., pentamer) or virus-like particle, as well as at least one selected from the group consisting of HPV2, -5, -6, -7, -8, -11, -16, -18, -26, -27, -28, -29, -30, -31, -32, -33, -34, -35, -38, -39, -40, -43, -44, -45, -51, -53, -56, -57, -58, -59, -61, -66, -67, -68, -69, -70, -73, -74, -77, -81, -82, -83, -85, -91 L1 virus-like particles, the content of which is an effective amount that can induce a protective immune response, respectively.

In particular embodiments, the vaccine contains the above-mentioned HPV52 L1 multimer (e.g., pentamer) or virus-like particle, as well as HPV6, -11, -16, -18, -26, -31, -33, -35, -39, -45, -51, -56, -58, -59, -68 and -73 L1 virus-like particles, the content of which is an effective amount that can induce a protective immune response, respectively.

In particular embodiments, the vaccine contains the above-mentioned HPV52 L1 multimer (e.g., pentamer) or virus-like particle, as well as HPV6, -11, -16, -18, -31, -33, -35, -39, -45 and -58 L1 virus-like particles, the content of which is an effective amount that can induce a protective immune response, respectively.

In particular embodiments, the vaccine contains the above-mentioned HPV52 L1 multimer (e.g., pentamer) or virus-like particle, as well as HPV6, -11, -16, -18 and -58 L1 virus-like particles, the content of which is an effective amount that can induce a protective immune response, respectively.

In particular embodiments, the vaccine contains the above-mentioned HPV52 L1 multimer (e.g., pentamer) or virus-like particle, as well as HPV16, -18 and -58 L1 virus-like particles, the content of which is an effective amount that can induce a protective immune response, respectively.

In particular embodiments, the vaccine contains the above-mentioned HPV52 L1 multimer (e.g., pentamer) or virus-like particle, as well as HPV16, -18 L1 virus-like particles, the content of which is an effective amount that can induce a protective immune response, respectively.

The present application relates to a novel vaccine that can further enhance the immune response, which comprises the above-mentioned HPV52 L1 multimer (e.g., pentamer) or virus-like particle as well as an adjuvant. Preferably, the adjuvant used is a human vaccine adjuvant.

According to some embodiments of the present application, provided is use of the above-mentioned modified HPV52 L1 protein, multimer (e.g., pentamer), virus-like particle and vaccine in the prevention of HPV infection or HPV infection-related diseases.

### Description and Explanation of Relevant Terms

According to the present application, the term “insect cell expression system” includes insect cell, recombinant baculovirus, recombinant Bacmid and expression vector. Among them, the insect cell is derived from a commercially available cell, the examples of which are listed here but not limited to: Sf9, Sf21, High Five.

According to the present application, examples of the term “wild-type HPV52 L1 protein” include, but are not limited to, L 1 protein corresponding to the sequence No. AEI61557.1 in NCBI database.

According to the present application, the term “excipient or carrier” refers to that selected from one or more of the following, including but not limited to, pH adjuster, surfactant and ionic strength enhancer. For example, the pH adjuster is for example but not limited to phosphate buffer. The surfactant includes cationic, anionic or nonionic surfactant, and is for example but not limited to polysorbate 80 (Tween-80). The ionic strength enhancer is for example but not limited to sodium chloride.

According to the present application, the term “adjuvant” refers to an adjuvant that can be applied clinically to the human body, including various adjuvants that have been approved and may be approved in the future.

According to the present application, the vaccine of the present application can be in a patient-acceptable form, including but not limited to oral administration or injection, preferably injection.

According to the present application, the vaccine of the present application is preferably used in a unit dosage form, wherein the dose of the optimally modified HPV52 L1 protein virus-like particle in the unit dosage form is 5 μg-80 μg, preferably 20 μg-40 μg.

### DESCRIPTION OF THE DRAWINGS

FIG. 1A and FIG. 1B show the expression identification of the wild-type HPV52 L1 and 28 mutants thereof in Example 4 of the present application in insect cells. The results show that the wild-type HPV52 L1 and 28 mutants thereof can all be expressed in insect cells. Lanes 1 to 15 of FIG. 1A represent wild-type HPV52L1, 52L1D447EΔC19, 52L1ΔN2, 52L1ΔN4, 52L1ΔN5, 52L1ΔN8, 52L1ΔN10, 52L1ΔN13, 52L1ΔN15, 52L1ΔN18, 52L1ΔN20, 52L1CS1, 52L1CS2, 52L1CS3 and 52L1CS4, respectively; Lanes 1 to 14 of FIG. 2A represent 52L1CS5, 52L1CS6, 52L1CS7, 52L1CS8, 52L1CS9, 52L1ΔN13CS1, 52L1ΔN13CS2, 52L1ΔN13CS3, 52L1NS1ΔC19, 52L1NS1ΔC25, 52L1NS2ΔC19, 52L1NS3ΔC19, 52L1NS4ΔC19 and 52L1ΔN14ΔC25, respectively.

FIGS. 2A to 2K show the dynamic light scattering analysis results of the wild-type HPV52L1, 52L1D447EΔC19, 52L1ΔN13, 52L1CS5, 52L1CS6, 52L1CS7, 52L1CS9, 52L1ΔN13CS1, 52L1ΔN13CS2, 52L1NS3ΔC19 and 52L1NS4ΔC19 mutant proteins obtained after purification in Example 5 of the present application. The results show that the hydraulic diameters of virus-like particles formed by wild-type HPV52L1, 52L1D447EΔC19, 52L1ΔN13, 52L1CS5, 52L1CS6, 52L1CS7, 52L1CS9, 52L1ΔN13CS1 and 52L1ΔN13CS2 recombinant proteins are 123.1 nm, 104.9 nm, 71.56 nm, 108.9 nm, 130.4 nm, 116 nm, 124 nm, 111.9 nm, 127.2 nm and 129.9 nm, respectively, and the percentage of particle assembly is 100%. 52L1NS3ΔC19 is not assembled. FIG. 2A represents wild-type HPV52L1; FIG. 2B represents 52L1D447EΔC19; FIG. 2C represents 52L1ΔN13; FIG. 2D represents 52L1CS5; FIG. 2E represents 52L1CS6; FIG. 2F represents 52L1CS7; FIG. 2G represents 52L1CS9; FIG. 2H represents 52L1ΔN13CS1; FIG. 2I represents 52L1ΔN13CS2; FIG. 2J represents 52L1NS3ΔC19; FIG. 2K represents 52L1NS4ΔC19.

FIG. 3A to FIG. 3I show the transmission electron microscopy observation results of the wild-type HPV52 L1, 52L1D447EΔC19, 52L1CS5, 52L1CS6, 52L1CS7, 52L1CS9, 52L1ΔN13CS1, 52L1ΔN13CS2 and 52L1NS4ΔC19 VLPs obtained after purification in Example 6 of the present application. A large number of virus-like particles with diameters of about 40-55 nm can be seen in the field. The particle size is consistent with the theoretical value and has good uniformity. Bar=100 nm. FIG. 3A represents wild-type HPV52L1; FIG. 3B represents 52L1D447EΔC19; FIG. 3C represents 52L1CS5; FIG. 3D represents 52L1CS6; FIG. 3E represents 52L1CS7; FIG. 3F represents 52L1CS9; FIG. 3G represents 52L1ΔN13CS1; FIG. 3H represents 52L1ΔN13CS2; FIG. 3I represents 52L1NS4ΔC19.

FIG. 4 shows the analysis of HPV52 neutralizing antibody titers in immune serum after inoculating mice with wild-type HPV52L1, 52L1D447EΔC19, 52L1ΔN13, 52L1CS5, 52L1CS6, 52L1CS7, 52L1CS9, 52L1ΔN13CS1, 52L1ΔN13CS2, 52L1NS3ΔC19 and 52L1NS4ΔC19 VLPs in Example 7 of the present application. ***: P<0.001.

### DETAILED DESCRIPTION OF THE INVENTION

The present application will be further illustrated by the non-limiting examples below. It is well known to those skilled in the art that many modifications can be made to the present application without departing from the spirit of the present application, and such modifications also fall within the scope of the present application. The following embodiments are only used to illustrate the present application and should not be regarded as limiting the scope of the present application, as the embodiments are necessarily diverse. The terms used in the present specification are intended only to describe particular embodiments but not as limitations. The scope of the present application has been defined in the appended claims.

Unless otherwise specified, all the technical and scientific terms used in the present specification have the same meaning as those generally understood by those skilled in the technical field to which the present application relates. Preferred methods and materials of the present application are described below, but any method and material similar or equivalent to the methods and materials described in the present specification can be used to implement or test the present application. Unless otherwise specified, the following experimental methods are conventional methods or methods described in product specifications. Unless otherwise specified, the experimental materials used are easily available from commercial companies. All published literatures referred to in the present specification are incorporated here by reference to reveal and illustrate the methods and/or materials in the published literatures.

### Example 1: Synthesis of the Gene of Mutated L1 Protein and Construction of Expression Vectors

The 28 mutated L1 proteins were as follows respectively:

1) 52L1D447EΔC19: The template was full-length HPV52 L1 gene (the sequence was as shown in SEQ ID NO. 1), and its corresponding amino acid sequence was the sequence No. AEI61557.1 in NCBI database (the sequence was as shown in SEQ ID NO. 30). The polynucleotide sequence encoding HPV52 L1D447EΔC19 was optimized by insect codons, constructed by deleting the nucleotides 1453-1509 of the HPV52 L1 gene backbone for insect cell codon optimization and mutating the nucleotides 1339-1341 from GAC to GAG (the amino acid sequence was as shown in SEQ ID NO. 2, and the nucleotide sequence was as shown in SEQ ID NO. 31), and synthesized by Shanghai Sangon Biotech Co., Ltd.

2) 52L1ΔN2: The template was HPV52 L1D447EΔC19 gene (the sequence was as shown in SEQ ID NO. 30). It was constructed by deleting the nucleotides 4-6 of HPV52 L1D447EΔC19 (the amino acid sequence was as shown in SEQ ID NO. 3, and the nucleotide sequence was as shown in SEQ ID NO. 32), and synthesized by Shanghai Sangon Biotech Co., Ltd.

3) 52L1ΔN4: The template was HPV52 L1D447EΔC19 gene (the sequence was as shown in SEQ ID NO. 30). It was constructed by deleting the nucleotides 4-12 of HPV52 L1D447EΔC19 (the amino acid sequence was as shown in SEQ ID NO. 4, and the nucleotide sequence was as shown in SEQ ID NO. 33), and synthesized by Shanghai Sangon Biotech Co., Ltd.

4) 52L1ΔN5: The template was HPV52 L1D447EΔC19 gene (the sequence was as shown in SEQ ID NO. 30). It was constructed by deleting the nucleotides 4-15 of HPV52 L1D447EΔC19 (the amino acid sequence was as shown in SEQ ID NO. 5, and the nucleotide sequence was as shown in SEQ ID NO. 34), and synthesized by Shanghai Sangon Biotech Co., Ltd.

5) 52L1ΔN8: The template was HPV52 L1D447EΔC19 gene (the sequence was as shown in SEQ ID NO. 30). It was constructed by deleting the nucleotides 4-24 of HPV52 L1D447EΔC19 (the amino acid sequence was as shown in SEQ ID NO. 6, and the nucleotide sequence was as shown in SEQ ID NO. 35), and synthesized by Shanghai Sangon Biotech Co., Ltd.

6) 52L1ΔN10: The template was HPV52 L1D447EΔC19 gene (the sequence was as shown in SEQ ID NO. 30). It was constructed by deleting the nucleotides 4-30 of HPV52 L1D447EΔC19 (the amino acid sequence was as shown in SEQ ID NO. 7, and the nucleotide sequence was as shown in SEQ ID NO. 36), and synthesized by Shanghai Sangon Biotech Co., Ltd.

7) 52L1ΔN13: The template was HPV52 L1D447EΔC19 gene (the sequence was as shown in SEQ ID NO. 30). It was constructed by deleting the nucleotides 4-39 of HPV52 L1D447EΔC19 (the amino acid sequence was as shown in SEQ ID NO. 8, and the nucleotide sequence was as shown in SEQ ID NO. 37), and synthesized by Shanghai Sangon Biotech Co., Ltd.

8) 52L1ΔN15: The template was HPV52 L1D447EΔC19 gene (the sequence was as shown in SEQ ID NO. 30). It was constructed by deleting the nucleotides 4-45 of HPV52 L1D447EΔC19 (the amino acid sequence was as shown in SEQ ID NO. 9, and the nucleotide sequence was as shown in SEQ ID NO. 38), and synthesized by Shanghai Sangon Biotech Co., Ltd.

9) 52L1ΔN18: The template was HPV52 L1D447EΔC19 gene (the sequence was as shown in SEQ ID NO. 30). It was constructed by deleting the nucleotides 4-54 of HPV52 L1D447EΔC19 (the amino acid sequence was as shown in SEQ ID NO. 10, and the nucleotide sequence was as shown in SEQ ID NO. 39), and synthesized by Shanghai Sangon Biotech Co., Ltd.

10) 52L1ΔN20: The template was HPV52 L1D447EΔC19 gene (the sequence was as shown in SEQ ID NO. 30). It was constructed by deleting the nucleotides 4-60 of HPV52 L1D447EΔC19 (the amino acid sequence was as shown in SEQ ID NO. 11, and the nucleotide sequence was as shown in SEQ ID NO. 40), and synthesized by Shanghai Sangon Biotech Co., Ltd.

11) 52L1CS1: The template was HPV52 L1D447EΔC19 gene (the sequence was as shown in SEQ ID NO. 30). It was constructed by mutating the nucleotides 1447-1449 of HPV52 L1D447EΔC19 from AAA to GGA and inserting the nucleotide sequence AAAGGTCCTGCATCGAGCGCTCCTAGAACGTCGACGGACGGCTCGGGAGTGGG ACGC after the nucleotide 1452 (the amino acid sequence was as shown in SEQ ID NO. 12, and the nucleotide sequence was as shown in SEQ ID NO. 41), and synthesized by Shanghai Sangon Biotech Co., Ltd.

12) 52L1CS2: The template was HPV52 L1D447EΔC19 gene (the sequence was as shown in SEQ ID NO. 30). It was constructed by mutating the nucleotides 1447-1449 of HPV52 L1D447EΔC19 from AAA to GGA and inserting the nucleotide sequence AAAGGTCCTGCATCGAGCGCTCCTAGAACGTCGACGGACGGCTCGGGAGTGGA CGGC after the nucleotide 1452 (the amino acid sequence was as shown in SEQ ID NO. 13, and the nucleotide sequence was as shown in SEQ ID NO. 42), and synthesized by Shanghai Sangon Biotech Co., Ltd.

13) 52L1CS3: The template was HPV52 L1D447EΔC19 gene (the sequence was as shown in SEQ ID NO. 30). It was constructed by mutating the nucleotides 1447-1449 of HPV52 L1D447EΔC19 from AAA to GGA and inserting the nucleotide sequence GGATCGCCTGCATCGAGCGCTCCTAGAACGTCGACGGACGGCTCGGGAGTGAA ACGC after the nucleotide 1452 (the amino acid sequence was as shown in SEQ ID NO. 14, and the nucleotide sequence was as shown in SEQ ID NO. 43), and synthesized by Shanghai Sangon Biotech Co., Ltd.

14) 52L1CS4: The template was HPV52 L1D447EΔC19 gene (the sequence was as shown in SEQ ID NO. 30). It was constructed by mutating the nucleotides 1447-1449 of HPV52 L1D447EΔC19 from AAA to GGA and inserting the nucleotide sequence GGATCGCCTGCATCGAGCGCTCCTAGAACGTCGACGGACGGCTCGGGAGTGGA CCGC after the nucleotide 1452 (the amino acid sequence was as shown in SEQ ID NO. 15, and the nucleotide sequence was as shown in SEQ ID NO. 44), and synthesized by Shanghai Sangon Biotech Co., Ltd.

52L1CS5: The template was HPV52 L1D447EΔC19 gene (the sequence was as shown in SEQ ID NO. 30). It was constructed by inserting the nucleotide sequence GCTGGTCCTGCCTCTTCCGCACCCGCGACTTCAACCGCTGCCGGCGGAGTTGGG TCG after the nucleotide 1452 of HPV52 L1D447EΔC19 (the amino acid sequence was as shown in SEQ ID NO. 16, and the nucleotide sequence was as shown in SEQ ID NO. 45), and synthesized by Shanghai Sangon Biotech Co., Ltd.

16) 52L1CS6: The template was HPV52 L1D447EΔC19 gene (the sequence was as shown in SEQ ID NO. 30). It was constructed by inserting the nucleotide sequence GAAGCTCCTGCCTCTTCCGCACCCGGTACTTCAACCGGCTCGAAAGCGGTTGCT GGA after the nucleotide 1452 of HPV52 L1D447EΔC19 (the amino acid sequence was as shown in SEQ ID NO. 17, and the nucleotide sequence was as shown in SEQ ID NO. 46), and synthesized by Shanghai Sangon Biotech Co., Ltd.

17) 52L1CS7: The template was HPV52 L1D447EΔC19 gene (the sequence was as shown in SEQ ID NO. 30). It was constructed by inserting the nucleotide sequence GCTGGTCCTGCTTCCTCAGCTCCAGCTACCTCAACCGACGGTTCTGGTGTGAAG CGC after the nucleotide 1452 of HPV52 L1D447EΔC19 (the amino acid sequence was as shown in SEQ ID NO. 18, and the nucleotide sequence was as shown in SEQ ID NO. 47), and synthesized by Shanghai Sangon Biotech Co., Ltd.

18) 52L1CS8: The template was HPV52 L1D447EΔC19 gene (the sequence was as shown in SEQ ID NO. 30). It was constructed by inserting the nucleotide sequence GCTGGTCCTGCTTCCTCAGCTCCACGTACCTCAACCGACGGTTCTGGTGTGAAG CGC after the nucleotide 1452 of HPV52 L1D447EΔC19 (the amino acid sequence was as shown in SEQ ID NO. 19, and the nucleotide sequence was as shown in SEQ ID NO. 48), and synthesized by Shanghai Sangon Biotech Co., Ltd.

19) 52L1CS9: The template was HPV52 L1D447EΔC19 gene (the sequence was as shown in SEQ ID NO. 30). It was constructed by mutating the nucleotides 1441-1443 of HPV52 L1D447EΔC19 from AGA to GGT, mutating the nucleotides 1447-1449 from AAA to GGC and inserting the nucleotide sequence TCGGGTCCTGCCTCGAGCGCCCCTAGAACGTCGACGGG TGGCTCGGCCGTGGGTAGC after the nucleotide 1452 of HPV52 L1D447EΔC19 (the amino acid sequence was as shown in SEQ ID NO. 20, and the nucleotide sequence was as shown in SEQ ID NO. 49), and synthesized by Shanghai Sangon Biotech Co., Ltd.

20) 52L1ΔN13CS1: The template was HPV52 L1ΔN13 gene (the sequence was as shown in SEQ ID NO. 37). It was constructed by mutating the nucleotides 1411-1416 of HPV52 L1ΔN13 from AAACTG to GGCTTG and inserting the nucleotide sequence TCGGGTCCTGCCTCGAGCGCCCCTAGAACGTCGACGGGTGGCTCGGCCGTGGGT AGC after the nucleotide 1416 (the amino acid sequence was as shown in SEQ ID NO. 21, and the nucleotide sequence was as shown in SEQ ID NO. 50), and synthesized by Shanghai Sangon Biotech Co., Ltd.

21) 52L1ΔN13CS2: The template was HPV52 L1ΔN13 gene (the sequence was as shown in SEQ ID NO. 37). It was constructed by mutating the nucleotides 1405-1407 of HPV52 L1ΔN13 from AGA to GGT, mutating the nucleotides 1411-1416 from AAACTG to GGCTTG and inserting the nucleotide sequence TCGGGTCCTGCCTCGAGCGCCCCTAGAACGTCGACGGGTGGCTCGGCC GTGGGTAGC after the nucleotide 1416 (the amino acid sequence was as shown in SEQ ID NO. 22, and the nucleotide sequence was as shown in SEQ ID NO. 51), and synthesized by Shanghai Sangon Biotech Co., Ltd.

22) 52L1ΔN13CS3: The template was HPV52 L1ΔN13 gene (the sequence was as shown in SEQ ID NO. 37). It was constructed by inserting the nucleotide sequence GCCGGTCCTGCCTCGAGCGCCCCTGCCACGTCGACGGCTGCGGGAGGCGTGGG TAGC after the nucleotide 1416 of HPV52 L1ΔN13 (the amino acid sequence was as shown in SEQ ID NO. 23, and the nucleotide sequence was as shown in SEQ ID NO. 52), and synthesized by Shanghai Sangon Biotech Co., Ltd.

23) 52L1NS1ΔC19: The template was HPV52 L1ΔN13 gene (the sequence was as shown in SEQ ID NO. 37). It was constructed by inserting the nucleotide sequence CCTAGCGAGGCTACC between the nucleotides 3/4 of HPV52 L1ΔN13 (the amino acid sequence was as shown in SEQ ID NO. 24, and the nucleotide sequence was as shown in SEQ ID NO. 53), and synthesized by Shanghai Sangon Biotech Co., Ltd.

24) 52L1NS1ΔC25: The template was HPV52 L1ΔN13 gene (the sequence was as shown in SEQ ID NO. 37). It was constructed by inserting the nucleotide sequence CCTAGCGAGGCTACC between the nucleotides 3/4 of HPV52 L1ΔN13 and deleting the nucleotides 1414-1431 (the amino acid sequence was as shown in SEQ ID NO. 25, and the nucleotide sequence was as shown in SEQ ID NO. 54), and synthesized by Shanghai Sangon Biotech Co., Ltd.

25) 52L1NS2ΔC19: The template was HPV52 L1ΔN13 gene (the sequence was as shown in SEQ ID NO. 37). It was constructed by inserting the nucleotide sequence TCCGAGCGT between the nucleotides 3/4 of HPV52 L1ΔN13 (the amino acid sequence was as shown in SEQ ID NO. 26, and the nucleotide sequence was as shown in SEQ ID NO. 55), and synthesized by Shanghai Sangon Biotech Co., Ltd.

26) 52L1NS3ΔC19: The template was HPV52 L1ΔN13 gene (the sequence was as shown in SEQ ID NO. 37). It was constructed by inserting the nucleotide sequence TCCGAG between the nucleotides 3/4 of HPV52 L1ΔN13 (the amino acid sequence was as shown in SEQ ID NO. 27, and the nucleotide sequence was as shown in SEQ ID NO. 56), and synthesized by Shanghai Sangon Biotech Co., Ltd.

27) 52L1NS4ΔC19: The template was HPV52 L1ΔN13 gene (the sequence was as shown in SEQ ID NO. 37). It was constructed by inserting the nucleotide sequence TCC between the nucleotides 3/4 of HPV52 L1ΔN13 (the amino acid sequence was as shown in SEQ ID NO. 28, and the nucleotide sequence was as shown in SEQ ID NO. 57), and synthesized by Shanghai Sangon Biotech Co., Ltd.

28) 52L1ΔN14ΔC25: The template was HPV52 L1ΔN13 gene (the sequence was as shown in SEQ ID NO. 37). It was constructed by deleting the nucleotides 4-6 and 1414-1431 of HPV52 L1ΔN13 (the amino acid sequence was as shown in SEQ ID NO. 29, and the nucleotide sequence was as shown in SEQ ID NO. 58), and synthesized by Shanghai Sangon Biotech Co., Ltd.

The EcoR I/BamH I restriction sites were used to digest the above-mentioned synthesized genes respectively, which were inserted into the commercial expression vector pFastBac1 (produced by Invitrogen) respectively to obtain recombinant expression vectors comprising the HPV52 L1 mutated genes, pFastBac1-52L1D447EΔC19, pFastBac1-52L1ΔN2, pFastBac1-52L1ΔN4, pFastBac1-52L1ΔN5, pFastBac1-52L1ΔN8, pFastBac1-52L1ΔN10, pFastBac1-52L1ΔN13, pFastBac1-52L1ΔN15, pFastBac1-52L1ΔN18, pFastBac1-52L1ΔN20, pFastBac1-52L1CS1, pFastBac1-52L1CS2, pFastBac1-52L1CS3, pFastBac1-52L1CS4, pFastBac1-52L1CS5, pFastBac1-52L1CS6, pFastBac1-52L1CS7, pFastBac1-52L1CS8, pFastBac1-52L1CS9, pFastBac1-52L1ΔN13CS1, pFastBac1-52L1ΔN13CS2, pFastBac1-52L1ΔN13CS3, pFastBac1-52L1NS 1 ΔC19, pFastBac1-52L1NS1ΔC25, pFastBac1-52L1NS2ΔC19, pFastBac1-52L1NS3ΔC19, pFastBac1-52L1NS4ΔC19 and pFastBac1-52L1ΔN14ΔC25.

The above-mentioned methods of enzyme digestion, ligation and construction of clones were all well known, for example, the patent CN 101293918 B.

### Example 2: Recombinant Bacmid and Recombinant Baculovirus Constructs of the HPV52 L1 Mutant Genes

The recombinant expression vectors comprising HPV52 L1 mutant gene, pFastBac1-52L1D447EΔC19, pFastBac1-52L1ΔN2, pFastBac1-52L1ΔN4, pFastBac1-52L1ΔN5, pFastBac1-52L1ΔN8, pFastBac1-52L1ΔN10, pFastBac1-52L1ΔN13, pFastBac1-52L1ΔN15, pFastBac1-52L1ΔN18, pFastBac1-52L1ΔN20, pFastBac1-52L1CS1, pFastBac1-52L1CS2, pFastBac1-52L1CS3, pFastBac1-52L1CS4, pFastBac1-52L1CS5, pFastBac1-52L1CS6, pFastBac1-52L1CS7, pFastBac1-52L1CS8, pFastBac1-52L1CS9, pFastBac1-52L1ΔN13CS1, pFastBac1-52L1ΔN13CS2, pFastBac1-52L1ΔN13C53, pFastBac1-52L1NS 1 ΔC19, pFastBac1-52L1NS1ΔC25, pFastBac1-52L1NS2ΔC19, pFastBac1-52L1NS3ΔC19, pFastBac1-52L1NS4ΔC19 and pFastBac1-52L1ΔN14ΔC25, were used to transform E. coli DH10Bac competent cells respectively, which were screened to obtain recombinant Bacmids. Then the recombinant Bacmids were used to transfect Sf9 insect cells to amplify recombinant baculoviruses within Sf9. Methods of screening of recombinant Bacmid and amplification of recombinant baculovirus were all well known, for example, the patent CN 101148661 B.

### Example 3: Expression of HPV52 L1 Mutant Genes in Sf9 Cells

519 cells were inoculated with the recombinant baculoviruses of 28 HPV52 L1 mutant genes to express the HPV52 L1 mutant proteins. After incubation at 27° C. for about 80 h, the fermentation broth was collected and centrifuged at 3,000 rpm for 15 min. The supernatant was discarded, and the cells were washed with PBS for use in expression identification and purification. Methods of infection and expression were publicly available, for example, the patent CN 101148661 B.

### Example 4: Expression Identification and Comparison of Expression Amounts of HPV52 L1 Mutant Proteins

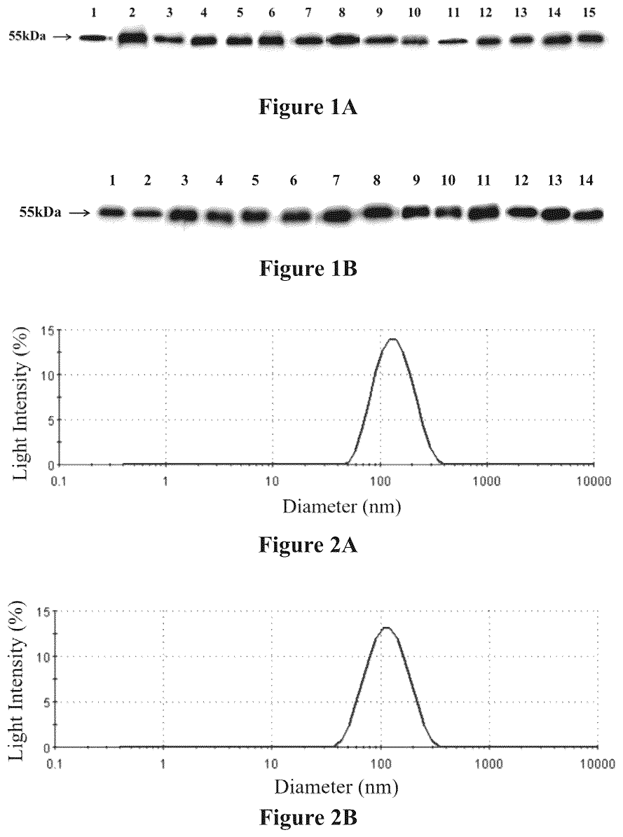

1×10⁶ cells expressing the different HPV52 L1 mutants and wild-type HPV52 L1 described in Example 3 respectively were collected and resuspended in 200 μl PBS solution. The cells were sonicated by ultrasonic disruption (Ningbo Scientz Ultrasonic Cell Disruptor, 2 #probe, 100 W, ultrasound 5 s, interval 7 s, total period 3 min) and centrifuged at a high speed of 13,000 rpm for 30 minutes. The lysed supernatant was collected and the total protein concentration in each lysed supernatant was detected by BCA assay. The lysed supernatant was uniformly diluted to 20 ng/μl with PBS. 2 μl of 6× loading buffer was added to 10 μl (i.e., 200 ng) of each diluted lysed supernatant. The samples were denatured at 75° C. for 8 min and subjected to SDS-PAGE electrophoresis and Western blot identification to compare the content of L1 protein (with a size of about 55 kDa) in the lysed supernatant of each mutant. The expression identification results of each mutant L1 protein were as shown in FIG. 1, and the comparison of expression amounts of each mutant L1 protein was as shown in Table 1. Methods of SDS-PAGE electrophoresis and Western blot identification were publicly available, for example, the patent CN 101148661 B.

Microtiter plates were coated with HPV52L1 monoclonal antibodies prepared by the inventor at 80 ng/well by overnight incubation at 4° C. The plate was blocked with 5% BSA-PBST at room temperature for 2 h and washed 3 times with PBST. The lysed supernatant was subjected to 2-fold serial dilution with PBS. The HPV52L1 VLP standard was also subjected to serial dilution from a concentration of 2 μg/ml to 0.0625 μg/ml. The diluted samples were added to the plate respectively at 100 μl per well and incubated at 37° C. for 1 h. The plate was washed 3 times with PBST, and 1:3000 diluted HPV52L1 rabbit polyclonal antibody was added at 100 μl per well and incubated at 37° C. for 1 h. The plate was washed 3 times with PBST, and 1:3000 diluted HRP-labeled goat anti-mouse IgG (1:3000 dilution, ZSGB-Bio Corporation) was added and incubated at 37° C. for 45 minutes. The plate was washed 5 times with PBST, and 100 μl of OPD substrate (Sigma) was added to each well for development at 37° C. for 5 minutes. The reaction was stopped with 50 μl of 2 M sulfuric acid, and the absorbance at 490 nm was determined. The concentrations of modified HPV52L1 proteins and wild-type HPV52L1 protein in the lysed supernatant were calculated according to the standard curve.

The results were as shown in Table 1. Different modifications had different effects on the expression level of HPV52L1 protein, among which the expression amount of some modified HPV52L1 proteins was increased, in particular 52L1ΔN13, 52L1CS7, 52L1CS9, 52L1ΔN13CS1, 52L1ΔN13CS2, 52 L1ΔN13CS3, 52 L1NS3ΔC19 and 52 L1NS4ΔC19, all with expression levels above 50 mg/L, much higher than the wild-type HPV52L1 protein.

TABLE 1
Analysis of expression amounts of HPV52 L1 mutant proteins

Expression amount (mg/L)

| Protein name | Batch 1 | Batch 2 | Batch 3 | Average |
|---|---|---|---|---|
| HPV52L1 | 3 | 3.5 | 5 | 3.83 |
| 52L1D447EΔC19 | 20 | 15 | 21 | 18.67 |
| 52L1ΔN2 | 5 | 3 | 4.5 | 4.17 |
| 52L1ΔN4 | 14 | 18 | 14 | 15.33 |
| 52L1ΔN5 | 15 | 11 | 16 | 14 |
| 52L1ΔN8 | 15 | 17 | 15 | 15.67 |
| 52L1ΔN10 | 21 | 20 | 20 | 20.33 |
| 52L1ΔN13 | 150 | 152 | 155 | 152.3 |
| 52L1ΔN15 | 4.5 | 5 | 4 | 4.5 |
| 52 L1ΔN18 | 5 | 4 | 4 | 4.33 |
| 52 L1ΔN20 | 16 | 13 | 14 | 14.33 |
| 52 L1CS1 | 25 | 28 | 24 | 25.67 |
| 52 L1CS2 | 31 | 29 | 33 | 31 |
| 52 L1CS3 | 42 | 38 | 39 | 39.67 |
| 52 L1CS4 | 40 | 44 | 43 | 42.33 |
| 52 L1CS5 | 43 | 40 | 36 | 42.33 |
| 52 L1CS6 | 40 | 35 | 48 | 41 |
| 52 L1CS7 | 75 | 73 | 79 | 75.67 |
| 52 L1CS8 | 23 | 21 | 20 | 21.33 |
| 52 L1CS9 | 60 | 67 | 63 | 63.33 |
| 52 L1ΔN13CS1 | 123 | 109 | 116 | 116 |
| 52 L1ΔN13CS2 | 108 | 112 | 105 | 108.33 |
| 52 L1ΔN13CS3 | 74 | 72 | 78 | 74.67 |
| 52 L1NS1ΔC19 | 20 | 23 | 25 | 22.67 |
| 52 L1NS1ΔC25 | 19 | 16 | 14 | 16.33 |
| 52 L1NS2ΔC19 | 10 | 7 | 15 | 10.67 |
| 52 L1NS3ΔC19 | 59 | 62 | 62 | 61 |
| 52 L1NS4ΔC19 | 104 | 108 | 99 | 103.67 |
| 52 L1ΔN14ΔC25 | 40 | 34 | 39 | 37.67 |

### Example 5: Purification and Dynamic Light Scattering Particle Size Analysis of L1 Mutant Proteins

An appropriate amount of cell fermentation broth of L1 mutants was collected and the cells were resuspended with PBS. PMSF was added to a final concentration of 1 mg/ml. The cells were ultrasonically disrupted (Ningbo Scientz Ultrasonic Cell Disruptor, 2 #probe, 200 W, ultrasound 5 s, interval 7 s, total period 10 min) and centrifuged at 13,000 rpm for 30 min. The supernatant was collected and diluted with PBS to 3-4 mg/mL. Saturated ammonium sulfate solution was added to the supernatant until ammonium sulfate saturation was 30%. The supernatant was let stand for precipitation at 4° C. for 1-2 hours and centrifuged at 13,000 rpm for 30 min. The precipitate was resuspended with an appropriate amount of resuspension buffer (20 mM Na₃PO₄, 50 mM DTT, 300 mM NaCl, pH 6.8) and stored on ice overnight. The chromatography purification step was performed at room temperature. The sample was filtered with 0.45 μm filter prior to chromatography and purified sequentially using SP-FF cation exchange chromatography and Q-HP anion exchange chromatography (100 mM NaCl, 20 mM Na₃PO₄, 10 mM DTT, pH 6.8 for loading). The purified product was assembled into VLPs in assembly buffer (500 mM NaCl, 2 mM CaCl₂, 2 mM MgCl·6H₂O, 20 mM HEPES, 0.01% Tween 80, pH 6.0) at 4° C. After 3 days of assembly, it was transferred into stabilization buffer (500 mM NaCl, 10 mM histidine, 0.01% Tween 80, pH 7.2) and stabilized at 4° C. for 2 days. The purification results showed that the purification yield of the modified 52L1 proteins was higher than that of the wild-type 52L1, in particular 52L1ΔN13, 52L1CS7, 52L1CS9, 52L1ΔN13CS1, 52L1ΔN13CS2, 52 L1ΔN13CS3, 52 L1NS3ΔC19 and 52 L1NS4ΔC19, with purification yields above 15 mg/L. The above purification methods were all publicly available, for example, the patents CN 101293918 B, CN 1976718 A, etc.

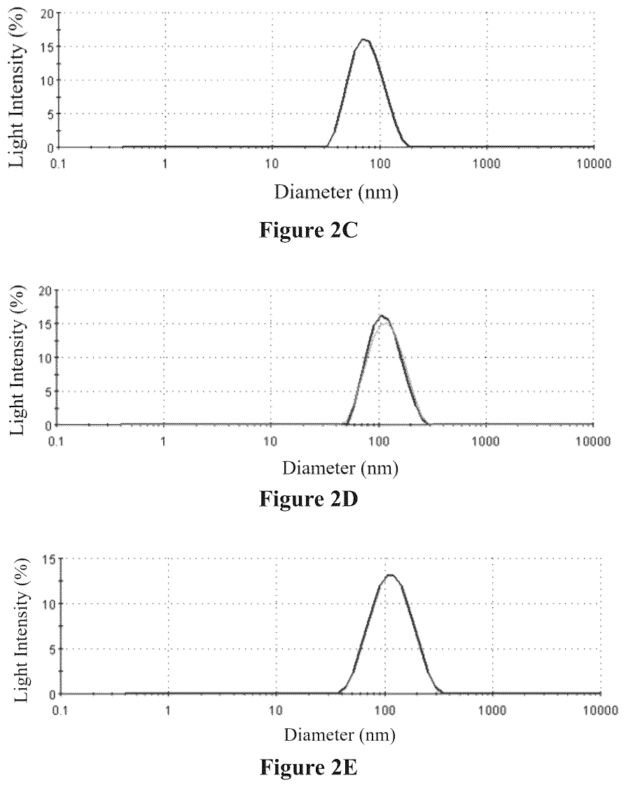

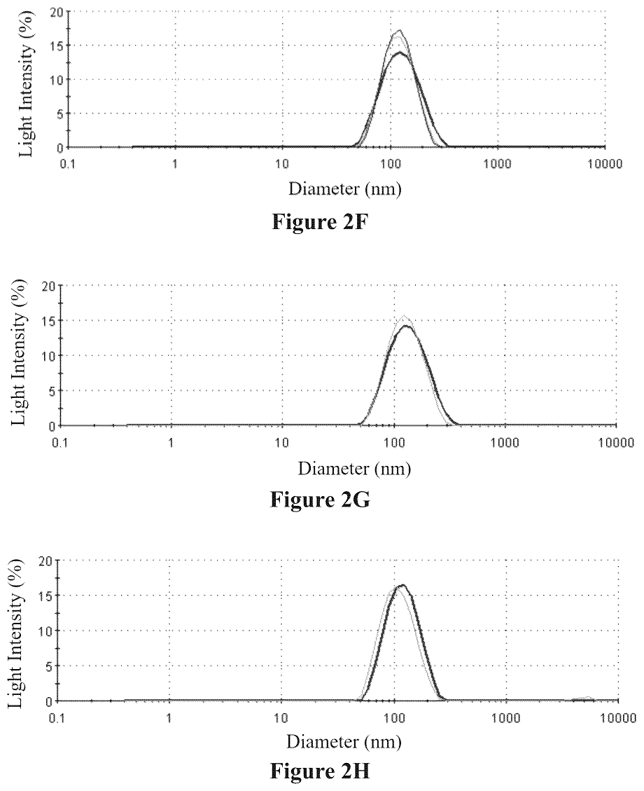

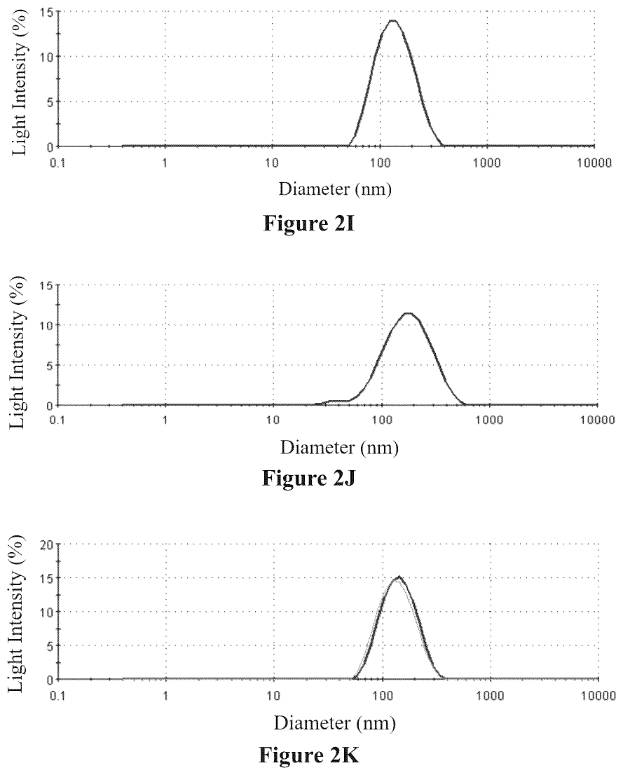

The purified protein solutions were subjected to DLS particle size analysis (Zetasizer Nano ZS 90 Dynamic Light Scatterer, Malvern), and the results were as shown in FIG. 2 and Table 2. Except for 52L1ΔN13, the hydraulic diameter of all the rest mutants was above 100 nm, close to that of HPV52L1. The hydraulic diameter of 52L1ΔN13 was only 71.56 nm, possibly suggesting its lower degree of assembly.

TABLE 2
DLS analysis of HPV52 L1 mutant proteins

| Protein name | Hydraulic diameter (nm) | PDI |
|---|---|---|
| HPV52L1 | 123.1 | 0.134 |
| 52L1D447EΔC19 | 104.9 | 0.142 |
| 52L1ΔN13 | 71.56 | 0.141 |
| 52L1CS5 | 108.9 | 0.126 |
| 52L1CS6 | 130.4 | 0.111 |
| 52L1CS7 | 116 | 0.135 |
| 52L1CS9 | 124 | 0.143 |
| 52L1ΔN13CS1 | 111.9 | 0.09 |
| 52L1ΔN13CS2 | 127.2 | 0.139 |
| 52L1NS3ΔC19 | 149.4 | 0.234 |
| 52L1NS4ΔC19 | 129.9 | 0.125 |

### Example 6: Transmission Electron Microscopy Observation of HPV52 L1 Mutant VLPs

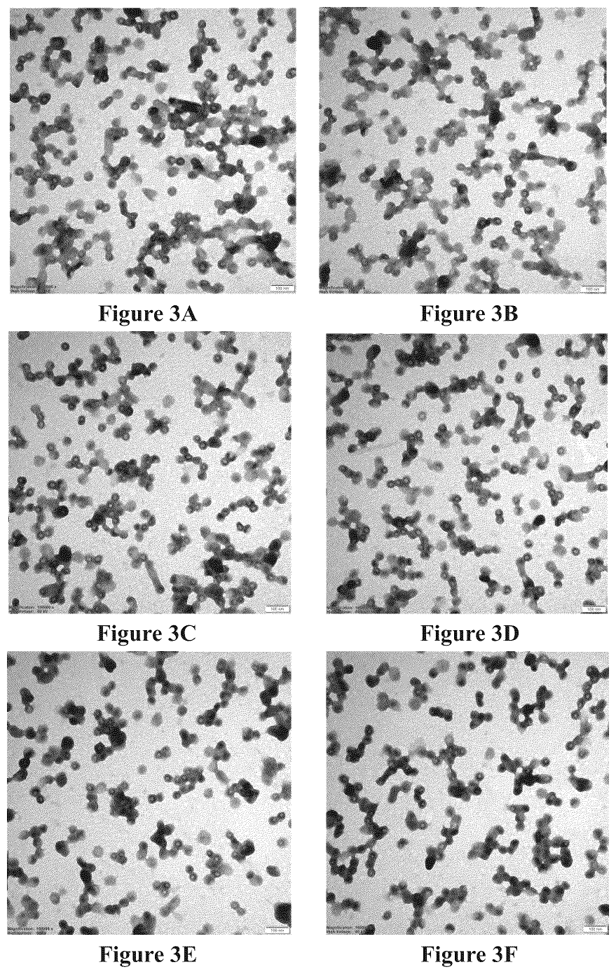

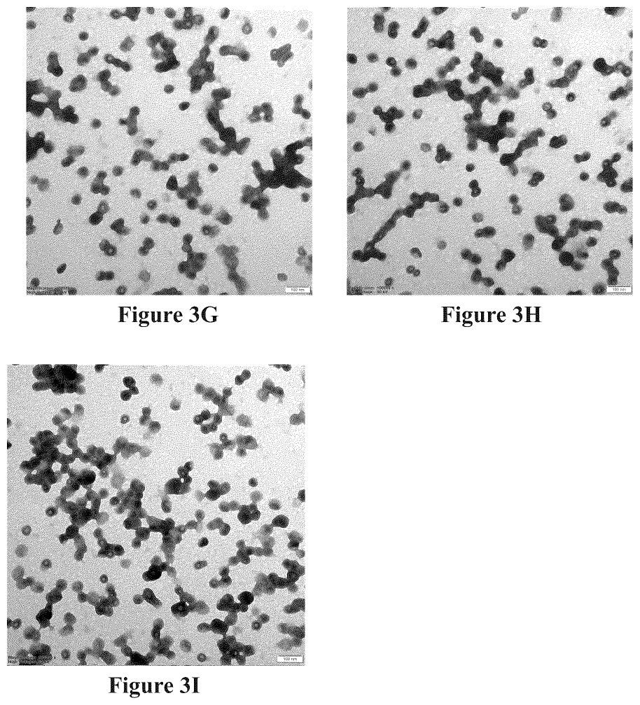

HPV52 L1 and mutant proteins thereof were purified and assembled respectively according to the chromatographic purification method described in Example 5. The assembled VLPs were prepared on copper mesh, stained with 1% uranium acetate, fully dried and then observed using JM-1400 electron microscope (Olympus). The transmission electron microscopy images of HPV52 L1, HPV52 L1D447EΔC19, HPV52 L1CS5, HPV52 L1CS6, HPV52 L1CS7, HPV52 L1CS9, HPV52 L1ΔN13CS1, HPV52 L1ΔN13CS2 and HPV52 L1NS4ΔC19 VLPs were as shown in FIGS. 3A-3I respectively. The diameter of all these mutants was between 40-55 nm. Methods of copper mesh preparation and electron microscopy observation were all publicly available, for example, the patent CN 101148661 B.

### Example 7: Immunization of Mice with HPV52 L1 Mutant VLPs and Determination of Neutralizing Antibody Titers

4-6 weeks old female BALB/c mice were randomly divided into groups of 5 mice and immunized with 0.1 μg VLP by intramuscular injection at Weeks 0, 2 and 4. Tail vein blood was collected 2 weeks after the third immunization and serum was isolated.

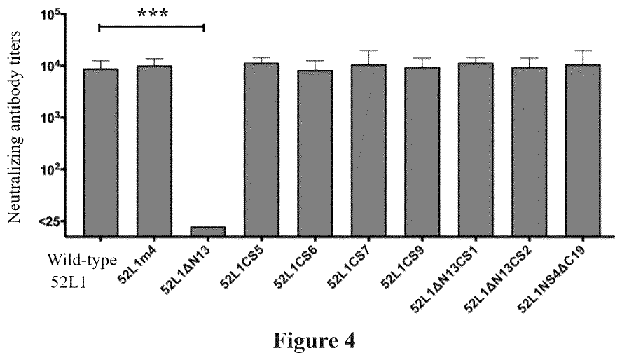

HPV52 pseudovirus was used to detect the neutralizing antibody titers in immune serum, and the results were as shown in Table 3 and FIG. 4. The neutralizing activity of immune serum caused by VLPs produced in insect cell expression systems, such as 52L1D447EΔC19, 52L1CS5, 52L1CS6, 52L1CS7, 52L1CS9, 52L1ΔN13CS1, 52L1ΔN13CS2 and 52L1NS4ΔC19, was comparable to that caused by HPV52L1, while 52L1ΔN13 immune serum had no neutralizing activity. Methods of pseudovirus preparation and pseudovirus neutralization experiments were all publicly available, for example, the patent CN 104418942A.

TABLE 3
Neutralizing antibody titers against HPV52 pseudovirus
induced by HPV52 L1 mutants in vivo in mice

| Antigen name | Average neutralizing antibody titer |
|---|---|
| HPV52L1 | 8960 |
| 52L1D447EΔC19 | 10240 |
| 52L1ΔN13 | <25 |
| 52L1CS5 | 11520 |
| 52L1CS6 | 8320 |
| 52L1CS7 | 10880 |
| 52L1CS9 | 9600 |
| 52L1ΔN13CS1 | 11520 |
| 52L1ΔN13CS2 | 9600 |
| 52L1NS4ΔC19 | 10880 |

In summary, the inventor has found that the mutants obtained by modification of HPV52L1 amino acid sequence have different expression levels. Their degree of assembly and immune activity can both be affected by the mutation modification in an irregular way. Therefore, it cannot be expected that HPV52L1 mutants with high expression level, effective assembly and good immune activity can be obtained by modification of the amino acid sequence. The optimally modified HPV52L1 mutants obtained by screening in the present application can be used in the formulation of multivalent HPV prophylactic vaccine and in the construction of broad-spectrum HPV prophylactic vaccine, and has good research and development prospects.

### Description of Sequences

SEQ ID NO. 1: HPV52L1
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHINNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKDYM
FWEVDLKEKFSADLDQFPLGRKFLLQAGLQARPKLKRPASSAPRTSTKKKKVKR
SEQ ID NO. 2: 52LID447EΔC19
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPKL
SEQ ID NO. 3: 52L1ΔN2
MVWRPSEATVYLPPVPVSKVVSTDEYVSRTSIYYYAGSSRLLTVGHPYFSIKNTSSG
NGKKVLVPKVSGLQYRVFRIKLPDPNKFGFPDTSFYNPETQRLVWACTGLEIGRGQ
PLGVGISGHPLLNKFDDTETSNKYAGKPGIDNRECLSMDYKQTQLCILGCKPPIGEH
WGKGTPCNNNSGNPGDCPPLQLINSVIQDGDMVDTGFGCMDFNTLQASKSDVPIDI
CSSVCKYPDYLQMASEPYGDSLFFFLRREQMFVRHFFNRAGTLGDPVPGDLYIQGS
NSGNTATVQSSAFFPTPSGSMVTSESQLFNKPYWLQRAQGHNNGICWGNQLFVTV
VDTTRSTNMTLCAEVKKESTYKNENFKEYLRHGEEFDLQFIFQLCKITLTADVMTYI
HKMDATILEDWQFGLTPPPSASLEDTYRFVTSTAITCQKNTPPKGKEDPLKEYMFW
EVDLKEKFSADLDQFPLGRKFLLQAGLQARPKL
SEQ ID NO. 4: 52L1ΔN4
MRPSEATVYLPPVPVSKVVSTDEYVSRTSIYYYAGSSRLLTVGHPYFSIKNTSSGNG
KKVLVPKVSGLQYRVFRIKLPDPNKFGFPDTSFYNPETQRLVWACTGLEIGRGQPLG
VGISGHPLLNKFDDTETSNKYAGKPGIDNRECLSMDYKQTQLCILGCKPPIGEHWG
KGTPCNNNSGNPGDCPPLQLINSVIQDGDMVDTGFGCMDENTLQASKSDVPIDICS
SVCKYPDYLQMASEPYGDSLFFFLRREQMFVRHFFNRAGTLGDPVPGDLYIQGSNS
GNTATVQSSAFFPTPSGSMVTSESQLFNKPYWLQRAQGHNNGICWGNQLFVTVVD
TTRSTNMTLCAEVKKESTYKNENFKEYLRHGEEFDLQFIFQLCKITLTADVMTYIHK
MDATILEDWQFGLTPPPSASLEDTYRFVTSTAITCQKNTPPKGKEDPLKEYMFWEV
DLKEKFSADLDQFPLGRKFLLQAGLQARPKL
SEQ ID NO. 5: 52LIΔN5
MPSEATVYLPPVPVSKVVSTDEYVSRTSIYYYAGSSRLLTVGHPYFSIKNTSSGNGK
KVLVPKVSGLQYRVFRIKLPDPNKFGFPDTSFYNPETQRLVWACTGLEIGRGQPLGV
GISGHPLLNKFDDTETSNKYAGKPGIDNRECLSMDYKQTQLCILGCKPPIGEHWGK
GTPCNNNSGNPGDCPPLQLINSVIQDGDMVDTGFGCMDENTLQASKSDVPIDICSS
VCKYPDYLQMASEPYGDSLFFFLRREQMFVRHFFNRAGTLGDPVPGDLYIQGSNSG
NTATVQSSAFFPTPSGSMVTSESQLFNKPYWLQRAQGHNNGICWGNQLFVTVVDT
TRSTNMTLCAEVKKESTYKNENFKEYLRHGEEFDLQFIFQLCKITLTADVMTYIHK
MDATILEDWQFGLTPPPSASLEDTYRFVTSTAITCQKNTPPKGKEDPLKEYMFWEV
DLKEKFSADLDQFPLGRKFLLQAGLQARPKL
SEQ ID NO. 6: 52L1ΔN8
MATVYLPPVPVSKVVSTDEYVSRTSIYYYAGSSRLLTVGHPYFSIKNTSSGNGKKVL
VPKVSGLQYRVFRIKLPDPNKFGFPDTSFYNPETQRLVWACTGLEIGRGQPLGVGIS
GHPLLNKFDDTETSNKYAGKPGIDNRECLSMDYKQTQLCILGCKPPIGEHWGKGTP
CNNNSGNPGDCPPLQLINSVIQDGDMVDTGFGCMDFNTLQASKSDVPIDICSSVCK
YPDYLQMASEPYGDSLFFFLRREQMFVRHFFNRAGTLGDPVPGDLYIQGSNSGNTA
TVQSSAFFPTPSGSMVTSESQLFNKPYWLQRAQGHNNGICWGNQLFVTVVDTTRS
TNMTLCAEVKKESTYKNENFKEYLRHGEEFDLQFIFQLCKITLTADVMTYIHKMDA
TILEDWQFGLTPPPSASLEDTYRFVTSTAITCQKNTPPKGKEDPLKEYMFWEVDLKE
KFSADLDQFPLGRKFLLQAGLQARPKL
SEQ ID NO. 7: 52LIΔN10
MVYLPPVPVSKVVSTDEYVSRTSIYYYAGSSRLLTVGHPYFSIKNTSSGNGKKVLVP
KVSGLQYRVFRIKLPDPNKFGFPDTSFYNPETQRLVWACTGLEIGRGQPLGVGISGH
PLLNKFDDTETSNKYAGKPGIDNRECLSMDYKQTQLCILGCKPPIGEHWGKGTPCN
NNSGNPGDCPPLQLINSVIQDGDMVDTGFGCMDFNTLQASKSDVPIDICSSVCKYP
DYLQMASEPYGDSLFFFLRREQMFVRHFFNRAGTLGDPVPGDLYIQGSNSGNTATV
QSSAFFPTPSGSMVTSESQLFNKPYWLQRAQGHNNGICWGNQLFVTVVDTTRSTN
MTLCAEVKKESTYKNENFKEYLRHGEEFDLQFIFQLCKITLTADVMTYIHKMDATIL
EDWQFGLTPPPSASLEDTYRFVTSTAITCQKNTPPKGKEDPLKEYMFWEVDLKEKF
SADLDQFPLGRKFLLQAGLQARPKL
SEQ ID NO. 8: 52L1ΔN13
MPPVPVSKVVSTDEYVSRTSIYYYAGSSRLLTVGHPYFSIKNTSSGNGKKVLVPKVS
GLQYRVFRIKLPDPNKFGFPDTSFYNPETQRLVWACTGLEIGRGQPLGVGISGHPLL
NKFDDTETSNKYAGKPGIDNRECLSMDYKQTQLCILGCKPPIGEHWGKGTPCNNNS
GNPGDCPPLQLINSVIQDGDMVDTGFGCMDFNTLQASKSDVPIDICSSVCKYPDYL
QMASEPYGDSLFFFLRREQMFVRHFFNRAGTLGDPVPGDLYIQGSNSGNTATVQSS
AFFPTPSGSMVTSESQLFNKPYWLQRAQGHNNGICWGNQLFVTVVDTTRSTNMTL
CAEVKKESTYKNENFKEYLRHGEEFDLQFIFQLCKITLTADVMTYIHKMDATILED
WQFGLTPPPSASLEDTYRFVTSTAITCQKNTPPKGKEDPLKEYMFWEVDLKEKFSA
DLDQFPLGRKFLLQAGLQARPKL
SEQ ID NO. 9: 52L1ΔN15
MVPVSKVVSTDEYVSRTSIYYYAGSSRLLTVGHPYFSIKNTSSGNGKKVLVPKVSGL
QYRVFRIKLPDPNKFGFPDTSFYNPETQRLVWACTGLEIGRGQPLGVGISGHPLLNK
FDDTETSNKYAGKPGIDNRECLSMDYKQTQLCILGCKPPIGEHWGKGTPCNNNSGN
PGDCPPLQLINSVIQDGDMVDTGFGCMDFNTLQASKSDVPIDICSSVCKYPDYLQM
ASEPYGDSLFFFLRREQMFVRHFFNRAGTLGDPVPGDLYIQGSNSGNTATVQSSAFF
PTPSGSMVTSESQLFNKPYWLQRAQGHNNGICWGNQLFVTVVDTTRSTNMTLCAE
VKKESTYKNENFKEYLRHGEEFDLQFIFQLCKITLTADVMTYIHKMDATILEDWQF
GLTPPPSASLEDTYRFVTSTAITCQKNTPPKGKEDPLKEYMFWEVDLKEKFSADLD
QFPLGRKFLLQAGLQARPKL
SEQ ID NO. 10: 52L1ΔN18
MSKVVSTDEYVSRTSIYYYAGSSRLLTVGHPYFSIKNTSSGNGKKVLVPKVSGLQYR
VFRIKLPDPNKFGFPDTSFYNPETQRLVWACTGLEIGRGQPLGVGISGHPLLNKFDD
TETSNKYAGKPGIDNRECLSMDYKQTQLCILGCKPPIGEHWGKGTPCNNNSGNPGD
CPPLQLINSVIQDGDMVDTGFGCMDFNTLQASKSDVPIDICSSVCKYPDYLQMASE
PYGDSLFFFLRREQMFVRHFFNRAGTLGDPVPGDLYIQGSNSGNTATVQSSAFFPTP
SGSMVTSESQLFNKPYWLQRAQGHNNGICWGNQLFVTVVDTTRSTNMTLCAEVK
KESTYKNENFKEYLRHGEEFDLQFIFQLCKITLTADVMTYIHKMDATILEDWQFGLT
PPPSASLEDTYRFVTSTAITCQKNTPPKGKEDPLKEYMFWEVDLKEKFSADLDQFPL
GRKFLLQAGLQARPKL
SEQ ID NO. 11: 52L1ΔN20
MVVSTDEYVSRTSIYYYAGSSRLLTVGHPYFSIKNTSSGNGKKVLVPKVSGLQYRVF
RIKLPDPNKFGFPDTSFYNPETQRLVWACTGLEIGRGQPLGVGISGHPLLNKFDDTE
TSNKYAGKPGIDNRECLSMDYKQTQLCILGCKPPIGEHWGKGTPCNNNSGNPGDCP
PLQLINSVIQDGDMVDTGFGCMDFNTLQASKSDVPIDICSSVCKYPDYLQMASEPY
GDSLFFFLRREQMFVRHFFNRAGTLGDPVPGDLYIQGSNSGNTATVQSSAFFPTPSG
SMVTSESQLFNKPYWLQRAQGHNNGICWGNQLFVTVVDTTRSTNMTLCAEVKKE
STYKNENFKEYLRHGEEFDLQFIFQLCKITLTADVMTYIHKMDATILEDWQFGLTPP
PSASLEDTYRFVTSTAITCQKNTPPKGKEDPLKEYMFWEVDLKEKFSADLDQFPLG
RKFLLQAGLQARPKL
SEQ ID NO. 12: 52L1CS1
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDENTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPGLKGPASSAPRTSTDGSGVGR
SEQ ID NO. 13: 52L1CS2
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPGLKGPASSAPRTSTDGSGVDG
SEQ ID NO. 14: 52L1CS3
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPGLGSPASSAPRTSTDGSGVKR
SEQ ID NO. 15: 52L1CS4
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPGLGSPASSAPRTSTDGSGVDR
SEQ ID NO. 16: 52L1CS5
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDENTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPKLAGPASSAPATSTAAGGVGS
SEQ ID NO. 17: 52L1CS6
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPKLEAPASSAPGTSTGSKAVAG
SEQ ID NO. 18: 52L1CS7
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDFNTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHINNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPKLAGPASSAPATSTDGSGVKR
SEQ ID NO. 19: 52L1CS8
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDENTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQARPKLAGPASSAPRTSTDGSGVKR
SEQ ID NO. 20: 52L1CS9
MSVWRPSEATVYLPPVPVSKVVSTDEYVSRTSIYYYAGSSRLLTVGHPYFSIKNTSS
GNGKKVLVPKVSGLQYRVFRIKLPDPNKFGFPDTSFYNPETQRLVWACTGLEIGRG
QPLGVGISGHPLLNKFDDTETSNKYAGKPGIDNRECLSMDYKQTQLCILGCKPPIGE
HWGKGTPCNNNSGNPGDCPPLQLINSVIQDGDMVDTGFGCMDENTLQASKSDVPI
DICSSVCKYPDYLQMASEPYGDSLFFFLRREQMFVRHFFNRAGTLGDPVPGDLYIQ
GSNSGNTATVQSSAFFPTPSGSMVTSESQLFNKPYWLQRAQGHNNGICWGNQLFV
TVVDTTRSTNMTLCAEVKKESTYKNENFKEYLRHGEEFDLQFIFQLCKITLTADVM
TYIHKMDATILEDWQFGLTPPPSASLEDTYRFVTSTAITCQKNTPPKGKEDPLKEYM
FWEVDLKEKFSADLDQFPLGRKFLLQAGLQAGPGLSGPASSAPRTSTGGSAVGS
SEQ ID NO. 21: 52L1ΔN13CS1
MPPVPVSKVVSTDEYVSRTSIYYYAGSSRLLTVGHPYFSIKNTSSGNGKKVLVPKVS
GLQYRVFRIKLPDPNKFGFPDTSFYNPETQRLVWACTGLEIGRGQPLGVGISGHPLL
NKFDDTETSNKYAGKPGIDNRECLSMDYKQTQLCILGCKPPIGEHWGKGTPCNNNS
GNPGDCPPLQLINSVIQDGDMVDTGFGCMDFNTLQASKSDVPIDICSSVCKYPDYL
QMASEPYGDSLFFFLRREQMFVRHFFNRAGTLGDPVPGDLYIQGSNSGNTATVQSS
AFFPTPSGSMVTSESQLFNKPYWLQRAQGHNNGICWGNQLFVTVVDTTRSTNMTL
CAEVKKESTYKNENFKEYLRHGEEFDLQFIFQLCKITLTADVMTYIHKMDATILED
WQFGLTPPPSASLEDTYRFVTSTAITCQKNTPPKGKEDPLKEYMFWEVDLKEKFSA
DLDQFPLGRKFLLQAGLQARPGLSGPASSAP481RTSTGGSAVGS
SEQ ID NO. 22: 52L1ΔN13CS2
MPPVPVSKVVSTDEYVSRTSIYYYAGSSRLLTVGHPYFSIKNTSSGNGKKVLVPKVS
GLQYRVFRIKLPDPNKFGFPDTSFYNPETQRLVWACTGLEIGRGQPLGVGISGHPLL
NKFDDTETSNKYAGKPGIDNRECLSMDYKQTQLCILGCKPPIGEHWGKGTPCNNNS
GNPGDCPPLQLINSVIQDGDMVDTGFGCMDFNTLQASKSDVPIDICSSVCKYPDYL
QMASEPYGDSLFFFLRREQMFVRHFFNRAGTLGDPVPGDLYIQGSNSGNTATVQSS
AFFPTPSGSMVTSESQLFNKPYWLQRAQGHNNGICWGNQLFVTVVDTTRSTNMTL
CAEVKKESTYKNENFKEYLRHGEEFDLQFIFQLCKITLTADVMTYIHKMDATILED
WQFGLTPPPSASLEDTYRFVTSTAITCQKNTPPKGKEDPLKEYMFWEVDLKEKFSA
DLDQFPLGRKFLLQAGLQAGPGLSGPASSAP481RTSTGGSAVGS
SEQ ID NO. 23: 52L1ΔN13CS3
MPPVPVSKVVSTDEYVSRTSIYYYAGSSRLLTVGHPYFSIKNTSSGNGKKVLVPKVS
GLQYRVFRIKLPDPNKFGFPDTSFYNPETQRLVWACTGLEIGRGQPLGVGISGHPLL
NKFDDTETSNKYAGKPGIDNRECLSMDYKQTQLCILGCKPPIGEHWGKGTPCNNNS
GNPGDCPPLQLINSVIQDGDMVDTGFGCMDFNTLQASKSDVPIDICSSVCKYPDYL
QMASEPYGDSLFFFLRREQMFVRHFFNRAGTLGDPVPGDLYIQGSNSGNTATVQSS
AFFPTPSGSMVTSESQLFNKPYWLQRAQGHNNGICWGNQLFVTVVDTTRSTNMTL
CAEVKKESTYKNENFKEYLRHGEEFDLQFIFQLCKITLTADVMTYIHKMDATILED
WQFGLTPPPSASLEDTYRFVTSTAITCQKNTPPKGKEDPLKEYMFWEVDLKEKFSA
DLDQFPLGRKFLLQAGLQARPKLAGPASSAP481ATSTAAGGVGS
SEQ ID NO. 24: 52L1NS1ΔC19
MPSEATPPVPVSKVVSTDEYVSRTSIYYYAGSSRLLTVGHPYFSIKNTSSGNGKKVL
VPKVSGLQYRVFRIKLPDPNKFGFPDTSFYNPETQRLVWACTGLEIGRGQPLGVGIS
GHPLLNKFDDTETSNKYAGKPGIDNRECLSMDYKQTQLCILGCKPPIGEHWGKGTP
CNNNSGNPGDCPPLQLINSVIQDGDMVDTGFGCMDFNTLQASKSDVPIDICSSVCK
YPDYLQMASEPYGDSLFFFLRREQMFVRHFFNRAGTLGDPVPGDLYIQGSNSGNTA
TVQSSAFFPTPSGSMVTSESQLFNKPYWLQRAQGHNNGICWGNQLFVTVVDTTRS
TNMTLCAEVKKESTYKNENFKEYLRHGEEFDLQFIFQLCKITLTADVMTYIHKMDA
TILEDWQFGLTPPPSASLEDTYRFVTSTAITCQKNTPPKGKEDPLKEYMFWEVDLKE
KFSADLDQFPLGRKFLLQAGLQARPKL
SEQ ID NO. 25: 52L1NS1ΔC25
MPSEATPPVPVSKVVSTDEYVSRTSIYYYAGSSRLLTVGHPYFSIKNTSSGNGKKVL
VPKVSGLQYRVFRIKLPDPNKFGFPDTSFYNPETQRLVWACTGLEIGRGQPLGVGIS
GHPLLNKFDDTETSNKYAGKPGIDNRECLSMDYKQTQLCILGCKPPIGEHWGKGTP
CNNNSGNPGDCPPLQLINSVIQDGDMVDTGFGCMDENTLQASKSDVPIDICSSVCK
YPDYLQMASEPYGDSLFFFLRREQMFVRHFFNRAGTLGDPVPGDLYIQGSNSGNTA
TVQSSAFFPTPSGSMVTSESQLFNKPYWLQRAQGHNNGICWGNQLFVTVVDTTRS
TNMTLCAEVKKESTYKNENFKEYLRHGEEFDLQFIFQLCKITLTADVMTYIHKMDA
TILEDWQFGLTPPPSASLEDTYRFVTSTAITCQKNTPPKGKEDPLKEYMFWEVDLKE
KFSADLDQFPLGRKFLLQAGL
SEQ ID NO. 26: 52L1NS2ΔC19
MSERPPVPVSKVVSTDEYVSRTSIYYYAGSSRLLTVGHPYFSIKNTSSGNGKKVLVP
KVSGLQYRVFRIKLPDPNKFGFPDTSFYNPETQRLVWACTGLEIGRGQPLGVGISGH
PLLNKFDDTETSNKYAGKPGIDNRECLSMDYKQTQLCILGCKPPIGEHWGKGTPCN
NNSGNPGDCPPLQLINSVIQDGDMVDTGFGCMDFNTLQASKSDVPIDICSSVCKYP
DYLQMASEPYGDSLFFFLRREQMFVRHFFNRAGTLGDPVPGDLYIQGSNSGNTATV
QSSAFFPTPSGSMVTSESQLFNKPYWLQRAQGHNNGICWGNQLFVTVVDTTRSTN
MTLCAEVKKESTYKNENFKEYLRHGEEFDLQFIFQLCKITLTADVMTYIHKMDATIL
EDWQFGLTPPPSASLEDTYRFVTSTAITCQKNTPPKGKEDPLKEYMFWEVDLKEKF
SADLDQFPLGRKFLLQAGLQARPKL
SEQ ID NO. 27: 52L1NS3ΔC19
MSEPPVPVSKVVSTDEYVSRTSIYYYAGSSRLLTVGHPYFSIKNTSSGNGKKVLVPK
VSGLQYRVFRIKLPDPNKFGFPDTSFYNPETQRLVWACTGLEIGRGQPLGVGISGHP
LLNKFDDTETSNKYAGKPGIDNRECLSMDYKQTQLCILGCKPPIGEHWGKGTPCNN
NSGNPGDCPPLQLINSVIQDGDMVDTGFGCMDFNTLQASKSDVPIDICSSVCKYPD
YLQMASEPYGDSLFFFLRREQMFVRHFFNRAGTLGDPVPGDLYIQGSNSGNTATVQ
SSAFFPTPSGSMVTSESQLFNKPYWLQRAQGHNNGICWGNQLFVTVVDTTRSTNM
TLCAEVKKESTYKNENFKEYLRHGEEFDLQFIFQLCKITLTADVMTYIHKMDATILE
DWQFGLTPPPSASLEDTYRFVTSTAITCQKNTPPKGKEDPLKEYMFWEVDLKEKFS
ADLDQFPLGRKFLLQAGLQARPKL
SEQ ID NO. 28: 52L1NS4ΔC19
MSPPVPVSKVVSTDEYVSRTSIYYYAGSSRLLTVGHPYFSIKNTSSGNGKKVLVPKV
SGLQYRVFRIKLPDPNKFGFPDTSFYNPETQRLVWACTGLEIGRGQPLGVGISGHPL
LNKFDDTETSNKYAGKPGIDNRECLSMDYKQTQLCILGCKPPIGEHWGKGTPCNN
NSGNPGDCPPLQLINSVIQDGDMVDTGFGCMDFNTLQASKSDVPIDICSSVCKYPD
YLQMASEPYGDSLFFFLRREQMFVRHFFNRAGTLGDPVPGDLYIQGSNSGNTATVQ
SSAFFPTPSGSMVTSESQLFNKPYWLQRAQGHNNGICWGNQLFVTVVDTTRSTNM
TLCAEVKKESTYKNENFKEYLRHGEEFDLQFIFQLCKITLTADVMTYIHKMDATILE
DWQFGLTPPPSASLEDTYRFVTSTAITCQKNTPPKGKEDPLKEYMFWEVDLKEKFS
ADLDQFPLGRKFLLQAGLQARPKL
SEQ ID NO. 29: 52L1ΔN14ΔC25
MPVPVSKVVSTDEYVSRTSIYYYAGSSRLLTVGHPYFSIKNTSSGNGKKVLVPKVSG
LQYRVFRIKLPDPNKFGFPDTSFYNPETQRLVWACTGLEIGRGQPLGVGISGHPLLN
KFDDTETSNKYAGKPGIDNRECLSMDYKQTQLCILGCKPPIGEHWGKGTPCNNNSG
NPGDCPPLQLINSVIQDGDMVDTGFGCMDFNTLQASKSDVPIDICSSVCKYPDYLQ
MASEPYGDSLFFFLRREQMFVRHFFNRAGTLGDPVPGDLYIQGSNSGNTATVQSSA
FFPTPSGSMVTSESQLFNKPYWLQRAQGHNNGICWGNQLFVTVVDTTRSTNMTLC
AEVKKESTYKNENFKEYLRHGEEFDLQFIFQLCKITLTADVMTYIHKMDATILEDW
QFGLTPPPSASLEDTYRFVTSTAITCQKNTPPKGKEDPLKEYMFWEVDLKEKFSADL
DQFPLGRKFLLQAGL
SEQ ID NO. 30: HPV52L1nt
ATGTCCGTGTGGCGGCCTAGTGAGGCCACTGTGTACCTGCCTCCTGTACCTGTCT
CTAAGGTTGTAAGCACTGATGAGTATGTGTCTCGCACAAGCATCTATTATTATGCA
GGCAGTTCTCGATTACTAACAGTAGGACATCCCTATTTTTCTATTAAAAACACCA
GTAGTGGTAATGGTAAAAAAGTTTTAGTTCCCAAGGTGTCTGGCCTGCAATACA
GGGTATTTAGAATTAAATTGCCGGACCCTAATAAATTTGGTTTTCCGGATACATCT
TTTTATAACCCAGAAACCCAAAGGTTGGTGTGGGCCTGTACAGGCTTGGAAATT
GGTAGGGGACAGCCTTTAGGTGTGGGTATTAGTGGGCATCCTTTATTAAACAAGT
TTGATGATACTGAAACCAGTAACAAATATGCTGGTAAACCTGGTATAGATAATAG
AGAATGTTTATCTATGGATTATAAGCAGACTCAGTTATGCATTTTAGGATGCAAAC
CTCCTATAGGTGAACATTGGGGTAAGGGAACCCCTTGTAATAATAATTCAGGAAA
TCCTGGGGATTGTCCTCCCCTACAACTCATTAACAGTGTAATACAGGATGGGGAC
ATGGTAGATACAGGATTTGGTTGCATGGATTTTAATACCTTGCAAGCTAGTAAAA
GTGATGTGCCCATTGATATATGTAGCAGTGTATGTAAGTATCCAGATTATTTGCAA
ATGGCTAGCGAGCCATATGGTGACAGTTTGTTCTTTTTTCTTAGACGTGAGCAAA
TGTTTGTTAGACACTTTTTTAATAGGGCTGGTACCTTAGGTGACCCTGTGCCAGG
TGATTTATATATACAAGGGTCTAACTCTGGCAATACTGCCACTGTACAAAGCAGT
GCTTTTTTTCCTACTCCTAGTGGTTCTATGGTAACCTCAGAATCCCAATTATTTAAT
AAACCGTACTGGTTACAACGTGCGCAGGGCCACAATAATGGCATATGTTGGGGC
AATCAGTTGTTTGTCACAGTTGTGGATACCACTCGTAGCACTAACATGACTTTAT
GTGCTGAAGTTAAAAAGGAAAGCACATATAAAAATGAAAATTTTAAGGAATACC
TTCGTCATGGCGAGGAATTTGATTTACAATTTATTTTTCAATTGTGCAAAATTACA
TTAACAGCTGATGTTATGACATACATTCATAAGATGGATGCCACTATTTTAGAGGA
CTGGCAATTTGGCCTTACCCCACCACCGTCTGCATCTTTGGAGGACACATACAGA
TTTGTAACTTCTACTGCTATAACTTGTCAAAAAAACACACCACCTAAAGGAAAG
GAAGATCCTTTAAAGGACTATATGTTTTGGGAGGTGGATTTAAAAGAAAAGTTTT
CTGCAGATTTAGATCAGTTTCCTTTAGGTAGGAAGTTTTTGTTACAGGCAGGGCT
ACAGGCTAGGCCCAAACTAAAACGCCCTGCATCATCAGCCCCACGTACCTCCAC
AAAGAAGAAAAAGGTTAAAAGGTAA
SEQ ID NO. 31: 52L1D447EΔC19nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTAGACCTAAACTGTAA
SEQ ID NO. 32: 52L1ΔN2nt
ATGGTGTGGCGTCCTTCCGAGGCTACTGTGTACTTGCCTCCAGTACCTGTTTCTA
AAGTGGTCTCCACTGATGAATACGTCTCACGTACCTCGATTTACTATTACGCTGGT
AGTTCAAGACTGTTGACAGTCGGCCACCCATACTTTTCTATCAAGAATACGTCCT
CAGGAAACGGTAAGAAGGTCCTTGTGCCGAAAGTTTCGGGTCTCCAATACCGCG
TCTTCCGTATCAAGCTGCCTGACCCCAACAAATTCGGCTTCCCAGATACTAGTTT
CTATAACCCAGAGACCCAGAGACTGGTGTGGGCCTGCACAGGACTCGAAATTGG
CAGGGGTCAACCTTTGGGCGTGGGAATCAGCGGTCACCCCCTTCTCAATAAGTT
CGACGACACAGAGACTTCTAACAAATACGCTGGTAAGCCAGGCATCGACAACC
GTGAATGCCTCTCCATGGATTACAAACAGACCCAACTGTGTATTCTGGGATGCAA
GCCGCCTATCGGTGAGCATTGGGGTAAAGGCACACCTTGCAACAATAACTCAGG
AAACCCAGGAGACTGCCCACCTTTGCAGCTTATCAACTCGGTTATTCAAGATGGT
GACATGGTCGACACTGGCTTTGGATGTATGGACTTCAATACTCTCCAGGCTTCCA
AGAGCGATGTCCCCATCGACATCTGCTCTTCCGTGTGTAAATACCCAGATTATCT
GCAAATGGCTTCAGAACCTTACGGAGACTCTCTGTTCTTCTTCTTGCGCAGGGA
GCAGATGTTCGTTCGTCACTTTTTCAACAGAGCCGGTACCTTGGGCGATCCTGTC
CCCGGAGACCTTTATATTCAAGGTTCCAACAGCGGTAACACAGCCACCGTGCAG
TCTTCCGCTTTCTTCCCAACTCCTTCAGGCAGCATGGTGACCAGTGAAAGCCAA
CTCTTTAATAAGCCTTACTGGTTGCAGAGGGCTCAAGGACACAACAATGGCATC
TGCTGGGGTAACCAGCTGTTCGTTACAGTCGTCGATACCACTCGTTCTACCAATA
TGACACTGTGCGCCGAGGTGAAGAAGGAATCCACATACAAAAACGAGAATTTC
AAGGAATACTTGCGTCACGGCGAGGAATTTGACCTTCAATTCATCTTCCAGCTCT
GCAAGATTACTCTCACCGCTGATGTTATGACATATATCCATAAGATGGACGCTACC
ATCCTGGAGGATTGGCAATTTGGACTGACTCCCCCACCCTCAGCTTCGTTGGAA
GACACCTACCGCTTCGTCACAAGTACTGCCATTACTTGTCAGAAGAACACTCCA
CCCAAGGGTAAGGAGGACCCACTTAAGGAGTACATGTTTTGGGAAGTGGATCTC
AAAGAGAAGTTCAGCGCCGACCTGGATCAATTTCCTCTGGGTCGTAAGTTCCTC
TTGCAAGCAGGACTGCAAGCTAGACCTAAACTGTAA
SEQ ID NO. 33: 52L1ΔN4nt
ATGCGTCCTTCCGAGGCTACTGTGTACTTGCCTCCAGTACCTGTTTCTAAAGTGG
TCTCCACTGATGAATACGTCTCACGTACCTCGATTTACTATTACGCTGGTAGTTCA
AGACTGTTGACAGTCGGCCACCCATACTTTTCTATCAAGAATACGTCCTCAGGAA
ACGGTAAGAAGGTCCTTGTGCCGAAAGTTTCGGGTCTCCAATACCGCGTCTTCC
GTATCAAGCTGCCTGACCCCAACAAATTCGGCTTCCCAGATACTAGTTTCTATAA
CCCAGAGACCCAGAGACTGGTGTGGGCCTGCACAGGACTCGAAATTGGCAGGG
GTCAACCTTTGGGCGTGGGAATCAGCGGTCACCCCCTTCTCAATAAGTTCGACG
ACACAGAGACTTCTAACAAATACGCTGGTAAGCCAGGCATCGACAACCGTGAAT
GCCTCTCCATGGATTACAAACAGACCCAACTGTGTATTCTGGGATGCAAGCCGC
CTATCGGTGAGCATTGGGGTAAAGGCACACCTTGCAACAATAACTCAGGAAACC
CAGGAGACTGCCCACCTTTGCAGCTTATCAACTCGGTTATTCAAGATGGTGACAT
GGTCGACACTGGCTTTGGATGTATGGACTTCAATACTCTCCAGGCTTCCAAGAGC
GATGTCCCCATCGACATCTGCTCTTCCGTGTGTAAATACCCAGATTATCTGCAAAT
GGCTTCAGAACCTTACGGAGACTCTCTGTTCTTCTTCTTGCGCAGGGAGCAGAT
GTTCGTTCGTCACTTTTTCAACAGAGCCGGTACCTTGGGCGATCCTGTCCCCGGA
GACCTTTATATTCAAGGTTCCAACAGCGGTAACACAGCCACCGTGCAGTCTTCC
GCTTTCTTCCCAACTCCTTCAGGCAGCATGGTGACCAGTGAAAGCCAACTCTTT
AATAAGCCTTACTGGTTGCAGAGGGCTCAAGGACACAACAATGGCATCTGCTGG
GGTAACCAGCTGTTCGTTACAGTCGTCGATACCACTCGTTCTACCAATATGACAC
TGTGCGCCGAGGTGAAGAAGGAATCCACATACAAAAACGAGAATTTCAAGGAA
TACTTGCGTCACGGCGAGGAATTTGACCTTCAATTCATCTTCCAGCTCTGCAAGA
TTACTCTCACCGCTGATGTTATGACATATATCCATAAGATGGACGCTACCATCCTG
GAGGATTGGCAATTTGGACTGACTCCCCCACCCTCAGCTTCGTTGGAAGACACC
TACCGCTTCGTCACAAGTACTGCCATTACTTGTCAGAAGAACACTCCACCCAAG
GGTAAGGAGGACCCACTTAAGGAGTACATGTTTTGGGAAGTGGATCTCAAAGAG
AAGTTCAGCGCCGACCTGGATCAATTTCCTCTGGGTCGTAAGTTCCTCTTGCAAG
CAGGACTGCAAGCTAGACCTAAACTGTAA
SEQ ID NO. 34: 52L1ΔN5nt
ATGCCTTCCGAGGCTACTGTGTACTTGCCTCCAGTACCTGTTTCTAAAGTGGTCT
CCACTGATGAATACGTCTCACGTACCTCGATTTACTATTACGCTGGTAGTTCAAG
ACTGTTGACAGTCGGCCACCCATACTTTTCTATCAAGAATACGTCCTCAGGAAAC
GGTAAGAAGGTCCTTGTGCCGAAAGTTTCGGGTCTCCAATACCGCGTCTTCCGTA
TCAAGCTGCCTGACCCCAACAAATTCGGCTTCCCAGATACTAGTTTCTATAACCC
AGAGACCCAGAGACTGGTGTGGGCCTGCACAGGACTCGAAATTGGCAGGGGTC
AACCTTTGGGCGTGGGAATCAGCGGTCACCCCCTTCTCAATAAGTTCGACGACA
CAGAGACTTCTAACAAATACGCTGGTAAGCCAGGCATCGACAACCGTGAATGCC
TCTCCATGGATTACAAACAGACCCAACTGTGTATTCTGGGATGCAAGCCGCCTAT
CGGTGAGCATTGGGGTAAAGGCACACCTTGCAACAATAACTCAGGAAACCCAG
GAGACTGCCCACCTTTGCAGCTTATCAACTCGGTTATTCAAGATGGTGACATGGT
CGACACTGGCTTTGGATGTATGGACTTCAATACTCTCCAGGCTTCCAAGAGCGAT
GTCCCCATCGACATCTGCTCTTCCGTGTGTAAATACCCAGATTATCTGCAAATGGC
TTCAGAACCTTACGGAGACTCTCTGTTCTTCTTCTTGCGCAGGGAGCAGATGTTC
GTTCGTCACTTTTTCAACAGAGCCGGTACCTTGGGCGATCCTGTCCCCGGAGAC
CTTTATATTCAAGGTTCCAACAGCGGTAACACAGCCACCGTGCAGTCTTCCGCTT
TCTTCCCAACTCCTTCAGGCAGCATGGTGACCAGTGAAAGCCAACTCTTTAATA
AGCCTTACTGGTTGCAGAGGGCTCAAGGACACAACAATGGCATCTGCTGGGGTA
ACCAGCTGTTCGTTACAGTCGTCGATACCACTCGTTCTACCAATATGACACTGTG
CGCCGAGGTGAAGAAGGAATCCACATACAAAAACGAGAATTTCAAGGAATACTT
GCGTCACGGCGAGGAATTTGACCTTCAATTCATCTTCCAGCTCTGCAAGATTACT
CTCACCGCTGATGTTATGACATATATCCATAAGATGGACGCTACCATCCTGGAGGA
TTGGCAATTTGGACTGACTCCCCCACCCTCAGCTTCGTTGGAAGACACCTACCG
CTTCGTCACAAGTACTGCCATTACTTGTCAGAAGAACACTCCACCCAAGGGTAA
GGAGGACCCACTTAAGGAGTACATGTTTTGGGAAGTGGATCTCAAAGAGAAGTT
CAGCGCCGACCTGGATCAATTTCCTCTGGGTCGTAAGTTCCTCTTGCAAGCAGG
ACTGCAAGCTAGACCTAAACTGTAA
SEQ ID NO. 35: 52L1ΔN8nt
ATGGCTACTGTGTACTTGCCTCCAGTACCTGTTTCTAAAGTGGTCTCCACTGATG
AATACGTCTCACGTACCTCGATTTACTATTACGCTGGTAGTTCAAGACTGTTGAC
AGTCGGCCACCCATACTTTTCTATCAAGAATACGTCCTCAGGAAACGGTAAGAA
GGTCCTTGTGCCGAAAGTTTCGGGTCTCCAATACCGCGTCTTCCGTATCAAGCTG
CCTGACCCCAACAAATTCGGCTTCCCAGATACTAGTTTCTATAACCCAGAGACCC
AGAGACTGGTGTGGGCCTGCACAGGACTCGAAATTGGCAGGGGTCAACCTTTG
GGCGTGGGAATCAGCGGTCACCCCCTTCTCAATAAGTTCGACGACACAGAGACT
TCTAACAAATACGCTGGTAAGCCAGGCATCGACAACCGTGAATGCCTCTCCATG
GATTACAAACAGACCCAACTGTGTATTCTGGGATGCAAGCCGCCTATCGGTGAG
CATTGGGGTAAAGGCACACCTTGCAACAATAACTCAGGAAACCCAGGAGACTG
CCCACCTTTGCAGCTTATCAACTCGGTTATTCAAGATGGTGACATGGTCGACACT
GGCTTTGGATGTATGGACTTCAATACTCTCCAGGCTTCCAAGAGCGATGTCCCCA
TCGACATCTGCTCTTCCGTGTGTAAATACCCAGATTATCTGCAAATGGCTTCAGA
ACCTTACGGAGACTCTCTGTTCTTCTTCTTGCGCAGGGAGCAGATGTTCGTTCGT
CACTTTTTCAACAGAGCCGGTACCTTGGGCGATCCTGTCCCCGGAGACCTTTATA
TTCAAGGTTCCAACAGCGGTAACACAGCCACCGTGCAGTCTTCCGCTTTCTTCC
CAACTCCTTCAGGCAGCATGGTGACCAGTGAAAGCCAACTCTTTAATAAGCCTT
ACTGGTTGCAGAGGGCTCAAGGACACAACAATGGCATCTGCTGGGGTAACCAG
CTGTTCGTTACAGTCGTCGATACCACTCGTTCTACCAATATGACACTGTGCGCCG
AGGTGAAGAAGGAATCCACATACAAAAACGAGAATTTCAAGGAATACTTGCGTC
ACGGCGAGGAATTTGACCTTCAATTCATCTTCCAGCTCTGCAAGATTACTCTCAC
CGCTGATGTTATGACATATATCCATAAGATGGACGCTACCATCCTGGAGGATTGGC
AATTTGGACTGACTCCCCCACCCTCAGCTTCGTTGGAAGACACCTACCGCTTCG
TCACAAGTACTGCCATTACTTGTCAGAAGAACACTCCACCCAAGGGTAAGGAGG
ACCCACTTAAGGAGTACATGTTTTGGGAAGTGGATCTCAAAGAGAAGTTCAGCG
CCGACCTGGATCAATTTCCTCTGGGTCGTAAGTTCCTCTTGCAAGCAGGACTGC
AAGCTAGACCTAAACTGTAA
SEQ ID NO. 36: 52L1ΔN10nt
ATGGTGTACTTGCCTCCAGTACCTGTTTCTAAAGTGGTCTCCACTGATGAATACG
TCTCACGTACCTCGATTTACTATTACGCTGGTAGTTCAAGACTGTTGACAGTCGG
CCACCCATACTTTTCTATCAAGAATACGTCCTCAGGAAACGGTAAGAAGGTCCTT
GTGCCGAAAGTTTCGGGTCTCCAATACCGCGTCTTCCGTATCAAGCTGCCTGACC
CCAACAAATTCGGCTTCCCAGATACTAGTTTCTATAACCCAGAGACCCAGAGACT
GGTGTGGGCCTGCACAGGACTCGAAATTGGCAGGGGTCAACCTTTGGGCGTGG
GAATCAGCGGTCACCCCCTTCTCAATAAGTTCGACGACACAGAGACTTCTAACA
AATACGCTGGTAAGCCAGGCATCGACAACCGTGAATGCCTCTCCATGGATTACA
AACAGACCCAACTGTGTATTCTGGGATGCAAGCCGCCTATCGGTGAGCATTGGG
GTAAAGGCACACCTTGCAACAATAACTCAGGAAACCCAGGAGACTGCCCACCT
TTGCAGCTTATCAACTCGGTTATTCAAGATGGTGACATGGTCGACACTGGCTTTG
GATGTATGGACTTCAATACTCTCCAGGCTTCCAAGAGCGATGTCCCCATCGACAT
CTGCTCTTCCGTGTGTAAATACCCAGATTATCTGCAAATGGCTTCAGAACCTTAC
GGAGACTCTCTGTTCTTCTTCTTGCGCAGGGAGCAGATGTTCGTTCGTCACTTTT
TCAACAGAGCCGGTACCTTGGGCGATCCTGTCCCCGGAGACCTTTATATTCAAGG
TTCCAACAGCGGTAACACAGCCACCGTGCAGTCTTCCGCTTTCTTCCCAACTCC
TTCAGGCAGCATGGTGACCAGTGAAAGCCAACTCTTTAATAAGCCTTACTGGTT
GCAGAGGGCTCAAGGACACAACAATGGCATCTGCTGGGGTAACCAGCTGTTCG
TTACAGTCGTCGATACCACTCGTTCTACCAATATGACACTGTGCGCCGAGGTGAA
GAAGGAATCCACATACAAAAACGAGAATTTCAAGGAATACTTGCGTCACGGCGA
GGAATTTGACCTTCAATTCATCTTCCAGCTCTGCAAGATTACTCTCACCGCTGAT
GTTATGACATATATCCATAAGATGGACGCTACCATCCTGGAGGATTGGCAATTTGG
ACTGACTCCCCCACCCTCAGCTTCGTTGGAAGACACCTACCGCTTCGTCACAAG
TACTGCCATTACTTGTCAGAAGAACACTCCACCCAAGGGTAAGGAGGACCCACT
TAAGGAGTACATGTTTTGGGAAGTGGATCTCAAAGAGAAGTTCAGCGCCGACCT
GGATCAATTTCCTCTGGGTCGTAAGTTCCTCTTGCAAGCAGGACTGCAAGCTAG
ACCTAAACTGTAA
SEQ ID NO. 37: 52L1ΔN13nt
ATGCCTCCAGTACCTGTTTCTAAAGTGGTCTCCACTGATGAATACGTCTCACGTA
CCTCGATTTACTATTACGCTGGTAGTTCAAGACTGTTGACAGTCGGCCACCCATA
CTTTTCTATCAAGAATACGTCCTCAGGAAACGGTAAGAAGGTCCTTGTGCCGAA
AGTTTCGGGTCTCCAATACCGCGTCTTCCGTATCAAGCTGCCTGACCCCAACAAA
TTCGGCTTCCCAGATACTAGTTTCTATAACCCAGAGACCCAGAGACTGGTGTGG
GCCTGCACAGGACTCGAAATTGGCAGGGGTCAACCTTTGGGCGTGGGAATCAG
CGGTCACCCCCTTCTCAATAAGTTCGACGACACAGAGACTTCTAACAAATACGC
TGGTAAGCCAGGCATCGACAACCGTGAATGCCTCTCCATGGATTACAAACAGAC
CCAACTGTGTATTCTGGGATGCAAGCCGCCTATCGGTGAGCATTGGGGTAAAGG
CACACCTTGCAACAATAACTCAGGAAACCCAGGAGACTGCCCACCTTTGCAGCT
TATCAACTCGGTTATTCAAGATGGTGACATGGTCGACACTGGCTTTGGATGTATG
GACTTCAATACTCTCCAGGCTTCCAAGAGCGATGTCCCCATCGACATCTGCTCTT
CCGTGTGTAAATACCCAGATTATCTGCAAATGGCTTCAGAACCTTACGGAGACTC
TCTGTTCTTCTTCTTGCGCAGGGAGCAGATGTTCGTTCGTCACTTTTTCAACAGA
GCCGGTACCTTGGGCGATCCTGTCCCCGGAGACCTTTATATTCAAGGTTCCAACA
GCGGTAACACAGCCACCGTGCAGTCTTCCGCTTTCTTCCCAACTCCTTCAGGCA
GCATGGTGACCAGTGAAAGCCAACTCTTTAATAAGCCTTACTGGTTGCAGAGGG
CTCAAGGACACAACAATGGCATCTGCTGGGGTAACCAGCTGTTCGTTACAGTCG
TCGATACCACTCGTTCTACCAATATGACACTGTGCGCCGAGGTGAAGAAGGAAT
CCACATACAAAAACGAGAATTTCAAGGAATACTTGCGTCACGGCGAGGAATTTG
ACCTTCAATTCATCTTCCAGCTCTGCAAGATTACTCTCACCGCTGATGTTATGACA
TATATCCATAAGATGGACGCTACCATCCTGGAGGATTGGCAATTTGGACTGACTC
CCCCACCCTCAGCTTCGTTGGAAGACACCTACCGCTTCGTCACAAGTACTGCCA
TTACTTGTCAGAAGAACACTCCACCCAAGGGTAAGGAGGACCCACTTAAGGAG
TACATGTTTTGGGAAGTGGATCTCAAAGAGAAGTTCAGCGCCGACCTGGATCAA
TTTCCTCTGGGTCGTAAGTTCCTCTTGCAAGCAGGACTGCAAGCTAGACCTAAA
CTGTAA
SEQ ID NO. 38: 52L1ΔN15nt
ATGGTACCTGTTTCTAAAGTGGTCTCCACTGATGAATACGTCTCACGTACCTCGA
TTTACTATTACGCTGGTAGTTCAAGACTGTTGACAGTCGGCCACCCATACTTTTCT
ATCAAGAATACGTCCTCAGGAAACGGTAAGAAGGTCCTTGTGCCGAAAGTTTCG
GGTCTCCAATACCGCGTCTTCCGTATCAAGCTGCCTGACCCCAACAAATTCGGCT
TCCCAGATACTAGTTTCTATAACCCAGAGACCCAGAGACTGGTGTGGGCCTGCA
CAGGACTCGAAATTGGCAGGGGTCAACCTTTGGGCGTGGGAATCAGCGGTCAC
CCCCTTCTCAATAAGTTCGACGACACAGAGACTTCTAACAAATACGCTGGTAAG
CCAGGCATCGACAACCGTGAATGCCTCTCCATGGATTACAAACAGACCCAACTG
TGTATTCTGGGATGCAAGCCGCCTATCGGTGAGCATTGGGGTAAAGGCACACCTT
GCAACAATAACTCAGGAAACCCAGGAGACTGCCCACCTTTGCAGCTTATCAACT
CGGTTATTCAAGATGGTGACATGGTCGACACTGGCTTTGGATGTATGGACTTCAA
TACTCTCCAGGCTTCCAAGAGCGATGTCCCCATCGACATCTGCTCTTCCGTGTGT
AAATACCCAGATTATCTGCAAATGGCTTCAGAACCTTACGGAGACTCTCTGTTCT
TCTTCTTGCGCAGGGAGCAGATGTTCGTTCGTCACTTTTTCAACAGAGCCGGTA
CCTTGGGCGATCCTGTCCCCGGAGACCTTTATATTCAAGGTTCCAACAGCGGTAA
CACAGCCACCGTGCAGTCTTCCGCTTTCTTCCCAACTCCTTCAGGCAGCATGGT
GACCAGTGAAAGCCAACTCTTTAATAAGCCTTACTGGTTGCAGAGGGCTCAAGG
ACACAACAATGGCATCTGCTGGGGTAACCAGCTGTTCGTTACAGTCGTCGATAC
CACTCGTTCTACCAATATGACACTGTGCGCCGAGGTGAAGAAGGAATCCACATA
CAAAAACGAGAATTTCAAGGAATACTTGCGTCACGGCGAGGAATTTGACCTTCA
ATTCATCTTCCAGCTCTGCAAGATTACTCTCACCGCTGATGTTATGACATATATCC
ATAAGATGGACGCTACCATCCTGGAGGATTGGCAATTTGGACTGACTCCCCCACC
CTCAGCTTCGTTGGAAGACACCTACCGCTTCGTCACAAGTACTGCCATTACTTGT
CAGAAGAACACTCCACCCAAGGGTAAGGAGGACCCACTTAAGGAGTACATGTT
TTGGGAAGTGGATCTCAAAGAGAAGTTCAGCGCCGACCTGGATCAATTTCCTCT
GGGTCGTAAGTTCCTCTTGCAAGCAGGACTGCAAGCTAGACCTAAACTGTAA
SEQ ID NO. 39: 52L1ΔN18nt
ATGTCTAAAGTGGTCTCCACTGATGAATACGTCTCACGTACCTCGATTTACTATTA
CGCTGGTAGTTCAAGACTGTTGACAGTCGGCCACCCATACTTTTCTATCAAGAAT
ACGTCCTCAGGAAACGGTAAGAAGGTCCTTGTGCCGAAAGTTTCGGGTCTCCAA
TACCGCGTCTTCCGTATCAAGCTGCCTGACCCCAACAAATTCGGCTTCCCAGATA
CTAGTTTCTATAACCCAGAGACCCAGAGACTGGTGTGGGCCTGCACAGGACTCG
AAATTGGCAGGGGTCAACCTTTGGGCGTGGGAATCAGCGGTCACCCCCTTCTCA
ATAAGTTCGACGACACAGAGACTTCTAACAAATACGCTGGTAAGCCAGGCATCG
ACAACCGTGAATGCCTCTCCATGGATTACAAACAGACCCAACTGTGTATTCTGGG
ATGCAAGCCGCCTATCGGTGAGCATTGGGGTAAAGGCACACCTTGCAACAATAA
CTCAGGAAACCCAGGAGACTGCCCACCTTTGCAGCTTATCAACTCGGTTATTCA
AGATGGTGACATGGTCGACACTGGCTTTGGATGTATGGACTTCAATACTCTCCAG
GCTTCCAAGAGCGATGTCCCCATCGACATCTGCTCTTCCGTGTGTAAATACCCAG
ATTATCTGCAAATGGCTTCAGAACCTTACGGAGACTCTCTGTTCTTCTTCTTGCG
CAGGGAGCAGATGTTCGTTCGTCACTTTTTCAACAGAGCCGGTACCTTGGGCGA
TCCTGTCCCCGGAGACCTTTATATTCAAGGTTCCAACAGCGGTAACACAGCCAC
CGTGCAGTCTTCCGCTTTCTTCCCAACTCCTTCAGGCAGCATGGTGACCAGTGA
AAGCCAACTCTTTAATAAGCCTTACTGGTTGCAGAGGGCTCAAGGACACAACAA
TGGCATCTGCTGGGGTAACCAGCTGTTCGTTACAGTCGTCGATACCACTCGTTCT
ACCAATATGACACTGTGCGCCGAGGTGAAGAAGGAATCCACATACAAAAACGA
GAATTTCAAGGAATACTTGCGTCACGGCGAGGAATTTGACCTTCAATTCATCTTC
CAGCTCTGCAAGATTACTCTCACCGCTGATGTTATGACATATATCCATAAGATGGA
CGCTACCATCCTGGAGGATTGGCAATTTGGACTGACTCCCCCACCCTCAGCTTCG
TTGGAAGACACCTACCGCTTCGTCACAAGTACTGCCATTACTTGTCAGAAGAAC
ACTCCACCCAAGGGTAAGGAGGACCCACTTAAGGAGTACATGTTTTGGGAAGTG
GATCTCAAAGAGAAGTTCAGCGCCGACCTGGATCAATTTCCTCTGGGTCGTAAG
TTCCTCTTGCAAGCAGGACTGCAAGCTAGACCTAAACTGTAA
SEQ ID NO. 40: 52L1ΔN20nt
ATGGTGGTCTCCACTGATGAATACGTCTCACGTACCTCGATTTACTATTACGCTGG
TAGTTCAAGACTGTTGACAGTCGGCCACCCATACTTTTCTATCAAGAATACGTCC
TCAGGAAACGGTAAGAAGGTCCTTGTGCCGAAAGTTTCGGGTCTCCAATACCGC
GTCTTCCGTATCAAGCTGCCTGACCCCAACAAATTCGGCTTCCCAGATACTAGTT
TCTATAACCCAGAGACCCAGAGACTGGTGTGGGCCTGCACAGGACTCGAAATTG
GCAGGGGTCAACCTTTGGGCGTGGGAATCAGCGGTCACCCCCTTCTCAATAAGT
TCGACGACACAGAGACTTCTAACAAATACGCTGGTAAGCCAGGCATCGACAACC
GTGAATGCCTCTCCATGGATTACAAACAGACCCAACTGTGTATTCTGGGATGCAA
GCCGCCTATCGGTGAGCATTGGGGTAAAGGCACACCTTGCAACAATAACTCAGG
AAACCCAGGAGACTGCCCACCTTTGCAGCTTATCAACTCGGTTATTCAAGATGGT
GACATGGTCGACACTGGCTTTGGATGTATGGACTTCAATACTCTCCAGGCTTCCA
AGAGCGATGTCCCCATCGACATCTGCTCTTCCGTGTGTAAATACCCAGATTATCT
GCAAATGGCTTCAGAACCTTACGGAGACTCTCTGTTCTTCTTCTTGCGCAGGGA
GCAGATGTTCGTTCGTCACTTTTTCAACAGAGCCGGTACCTTGGGCGATCCTGTC
CCCGGAGACCTTTATATTCAAGGTTCCAACAGCGGTAACACAGCCACCGTGCAG
TCTTCCGCTTTCTTCCCAACTCCTTCAGGCAGCATGGTGACCAGTGAAAGCCAA
CTCTTTAATAAGCCTTACTGGTTGCAGAGGGCTCAAGGACACAACAATGGCATC
TGCTGGGGTAACCAGCTGTTCGTTACAGTCGTCGATACCACTCGTTCTACCAATA
TGACACTGTGCGCCGAGGTGAAGAAGGAATCCACATACAAAAACGAGAATTTC
AAGGAATACTTGCGTCACGGCGAGGAATTTGACCTTCAATTCATCTTCCAGCTCT
GCAAGATTACTCTCACCGCTGATGTTATGACATATATCCATAAGATGGACGCTACC
ATCCTGGAGGATTGGCAATTTGGACTGACTCCCCCACCCTCAGCTTCGTTGGAA
GACACCTACCGCTTCGTCACAAGTACTGCCATTACTTGTCAGAAGAACACTCCA
CCCAAGGGTAAGGAGGACCCACTTAAGGAGTACATGTTTTGGGAAGTGGATCTC
AAAGAGAAGTTCAGCGCCGACCTGGATCAATTTCCTCTGGGTCGTAAGTTCCTC
TTGCAAGCAGGACTGCAAGCTAGACCTAAACTGTAA
SEQ ID NO. 41: 52L1CS1nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTCGTCCTGGACTGAAAGGTCCTGCATCGAGCG
CTCCTAGAACGTCGACGGACGGCTCGGGAGTGGGACGCTAA
SEQ ID NO. 42: 52L1CS2nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTCGTCCTGGACTGAAAGGTCCTGCATCGAGCG
CTCCTAGAACGTCGACGGACGGCTCGGGAGTGGACGGCTAA
SEQ ID NO. 43: 52L1CS3nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTCGTCCTGGACTGGGATCGCCTGCATCGAGCGC
TCCTAGAACGTCGACGGACGGCTCGGGAGTGAAACGCTAA
SEQ ID NO. 44: 52L1CS4nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTCGTCCTGGACTGGGATCGCCTGCATCGAGCGC
TCCTAGAACGTCGACGGACGGCTCGGGAGTGGACCGCTAA
SEQ ID NO. 45: 52L1CS5nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTAGACCTAAACTGGCTGGTCCTGCCTCTTCCGC
ACCCGCGACTTCAACCGCTGCCGGCGGAGTTGGGTCGTAA
SEQ ID NO. 46: 52L1CS6nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTAGACCTAAACTGGAAGCTCCTGCCTCTTCCGC
ACCCGGTACTTCAACCGGCTCGAAAGCGGTTGCTGGATAA
SEQ ID NO. 47: 52L1CS7nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTAGACCTAAACTGGCTGGTCCTGCTTCCTCAGC
TCCAGCTACCTCAACCGACGGTTCTGGTGTGAAGCGCTAA
SEQ ID NO. 48: 52L1CS8nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCTAGACCTAAACTGGCTGGTCCTGCTTCCTCAGC
TCCACGTACCTCAACCGACGGTTCTGGTGTGAAGCGCTAA
SEQ ID NO. 49: 52L1CS9nt
ATGTCCGTGTGGCGTCCTTCCGAGGCTACTGTGTACTTGCCTCCAGTACCTGTTT
CTAAAGTGGTCTCCACTGATGAATACGTCTCACGTACCTCGATTTACTATTACGCT
GGTAGTTCAAGACTGTTGACAGTCGGCCACCCATACTTTTCTATCAAGAATACGT
CCTCAGGAAACGGTAAGAAGGTCCTTGTGCCGAAAGTTTCGGGTCTCCAATACC
GCGTCTTCCGTATCAAGCTGCCTGACCCCAACAAATTCGGCTTCCCAGATACTAG
TTTCTATAACCCAGAGACCCAGAGACTGGTGTGGGCCTGCACAGGACTCGAAAT
TGGCAGGGGTCAACCTTTGGGCGTGGGAATCAGCGGTCACCCCCTTCTCAATAA
GTTCGACGACACAGAGACTTCTAACAAATACGCTGGTAAGCCAGGCATCGACAA
CCGTGAATGCCTCTCCATGGATTACAAACAGACCCAACTGTGTATTCTGGGATGC
AAGCCGCCTATCGGTGAGCATTGGGGTAAAGGCACACCTTGCAACAATAACTCA
GGAAACCCAGGAGACTGCCCACCTTTGCAGCTTATCAACTCGGTTATTCAAGAT
GGTGACATGGTCGACACTGGCTTTGGATGTATGGACTTCAATACTCTCCAGGCTT
CCAAGAGCGATGTCCCCATCGACATCTGCTCTTCCGTGTGTAAATACCCAGATTA
TCTGCAAATGGCTTCAGAACCTTACGGAGACTCTCTGTTCTTCTTCTTGCGCAGG
GAGCAGATGTTCGTTCGTCACTTTTTCAACAGAGCCGGTACCTTGGGCGATCCT
GTCCCCGGAGACCTTTATATTCAAGGTTCCAACAGCGGTAACACAGCCACCGTG
CAGTCTTCCGCTTTCTTCCCAACTCCTTCAGGCAGCATGGTGACCAGTGAAAGC
CAACTCTTTAATAAGCCTTACTGGTTGCAGAGGGCTCAAGGACACAACAATGGC
ATCTGCTGGGGTAACCAGCTGTTCGTTACAGTCGTCGATACCACTCGTTCTACCA
ATATGACACTGTGCGCCGAGGTGAAGAAGGAATCCACATACAAAAACGAGAATT
TCAAGGAATACTTGCGTCACGGCGAGGAATTTGACCTTCAATTCATCTTCCAGCT
CTGCAAGATTACTCTCACCGCTGATGTTATGACATATATCCATAAGATGGACGCTA
CCATCCTGGAGGATTGGCAATTTGGACTGACTCCCCCACCCTCAGCTTCGTTGGA
AGACACCTACCGCTTCGTCACAAGTACTGCCATTACTTGTCAGAAGAACACTCC
ACCCAAGGGTAAGGAGGACCCACTTAAGGAGTACATGTTTTGGGAAGTGGATCT
CAAAGAGAAGTTCAGCGCCGACCTGGATCAATTTCCTCTGGGTCGTAAGTTCCT
CTTGCAAGCAGGACTGCAAGCGGGTCCTGGCTTGTCGGGTCCTGCCTCGAGCGC
CCCTAGAACGTCGACGGGTGGCTCGGCCGTGGGTAGCTAA
SEQ ID NO. 50: 52L1ΔN13CS1nt
ATGCCTCCAGTACCTGTTTCTAAAGTGGTCTCCACTGATGAATACGTCTCACGTA
CCTCGATTTACTATTACGCTGGTAGTTCAAGACTGTTGACAGTCGGCCACCCATA
CTTTTCTATCAAGAATACGTCCTCAGGAAACGGTAAGAAGGTCCTTGTGCCGAA
AGTTTCGGGTCTCCAATACCGCGTCTTCCGTATCAAGCTGCCTGACCCCAACAAA
TTCGGCTTCCCAGATACTAGTTTCTATAACCCAGAGACCCAGAGACTGGTGTGG
GCCTGCACAGGACTCGAAATTGGCAGGGGTCAACCTTTGGGCGTGGGAATCAG
CGGTCACCCCCTTCTCAATAAGTTCGACGACACAGAGACTTCTAACAAATACGC
TGGTAAGCCAGGCATCGACAACCGTGAATGCCTCTCCATGGATTACAAACAGAC
CCAACTGTGTATTCTGGGATGCAAGCCGCCTATCGGTGAGCATTGGGGTAAAGG
CACACCTTGCAACAATAACTCAGGAAACCCAGGAGACTGCCCACCTTTGCAGCT
TATCAACTCGGTTATTCAAGATGGTGACATGGTCGACACTGGCTTTGGATGTATG
GACTTCAATACTCTCCAGGCTTCCAAGAGCGATGTCCCCATCGACATCTGCTCTT
CCGTGTGTAAATACCCAGATTATCTGCAAATGGCTTCAGAACCTTACGGAGACTC
TCTGTTCTTCTTCTTGCGCAGGGAGCAGATGTTCGTTCGTCACTTTTTCAACAGA
GCCGGTACCTTGGGCGATCCTGTCCCCGGAGACCTTTATATTCAAGGTTCCAACA
GCGGTAACACAGCCACCGTGCAGTCTTCCGCTTTCTTCCCAACTCCTTCAGGCA
GCATGGTGACCAGTGAAAGCCAACTCTTTAATAAGCCTTACTGGTTGCAGAGGG
CTCAAGGACACAACAATGGCATCTGCTGGGGTAACCAGCTGTTCGTTACAGTCG
TCGATACCACTCGTTCTACCAATATGACACTGTGCGCCGAGGTGAAGAAGGAAT
CCACATACAAAAACGAGAATTTCAAGGAATACTTGCGTCACGGCGAGGAATTTG
ACCTTCAATTCATCTTCCAGCTCTGCAAGATTACTCTCACCGCTGATGTTATGACA
TATATCCATAAGATGGACGCTACCATCCTGGAGGATTGGCAATTTGGACTGACTC
CCCCACCCTCAGCTTCGTTGGAAGACACCTACCGCTTCGTCACAAGTACTGCCA
TTACTTGTCAGAAGAACACTCCACCCAAGGGTAAGGAGGACCCACTTAAGGAG
TACATGTTTTGGGAAGTGGATCTCAAAGAGAAGTTCAGCGCCGACCTGGATCAA
TTTCCTCTGGGTCGTAAGTTCCTCTTGCAAGCAGGACTGCAAGCGAGACCTGGC
TTGTCGGGTCCTGCCTCGAGCGCCCCTAGAACGTCGACGGGTGGCTCGGCCGTG
GGTAGCTAA
SEQ ID NO. 51: 52L1ΔN13CS2nt
ATGCCTCCAGTACCTGTTTCTAAAGTGGTCTCCACTGATGAATACGTCTCACGTA
CCTCGATTTACTATTACGCTGGTAGTTCAAGACTGTTGACAGTCGGCCACCCATA
CTTTTCTATCAAGAATACGTCCTCAGGAAACGGTAAGAAGGTCCTTGTGCCGAA
AGTTTCGGGTCTCCAATACCGCGTCTTCCGTATCAAGCTGCCTGACCCCAACAAA
TTCGGCTTCCCAGATACTAGTTTCTATAACCCAGAGACCCAGAGACTGGTGTGG
GCCTGCACAGGACTCGAAATTGGCAGGGGTCAACCTTTGGGCGTGGGAATCAG
CGGTCACCCCCTTCTCAATAAGTTCGACGACACAGAGACTTCTAACAAATACGC
TGGTAAGCCAGGCATCGACAACCGTGAATGCCTCTCCATGGATTACAAACAGAC
CCAACTGTGTATTCTGGGATGCAAGCCGCCTATCGGTGAGCATTGGGGTAAAGG
CACACCTTGCAACAATAACTCAGGAAACCCAGGAGACTGCCCACCTTTGCAGCT
TATCAACTCGGTTATTCAAGATGGTGACATGGTCGACACTGGCTTTGGATGTATG
GACTTCAATACTCTCCAGGCTTCCAAGAGCGATGTCCCCATCGACATCTGCTCTT
CCGTGTGTAAATACCCAGATTATCTGCAAATGGCTTCAGAACCTTACGGAGACTC
TCTGTTCTTCTTCTTGCGCAGGGAGCAGATGTTCGTTCGTCACTTTTTCAACAGA
GCCGGTACCTTGGGCGATCCTGTCCCCGGAGACCTTTATATTCAAGGTTCCAACA
GCGGTAACACAGCCACCGTGCAGTCTTCCGCTTTCTTCCCAACTCCTTCAGGCA
GCATGGTGACCAGTGAAAGCCAACTCTTTAATAAGCCTTACTGGTTGCAGAGGG
CTCAAGGACACAACAATGGCATCTGCTGGGGTAACCAGCTGTTCGTTACAGTCG
TCGATACCACTCGTTCTACCAATATGACACTGTGCGCCGAGGTGAAGAAGGAAT
CCACATACAAAAACGAGAATTTCAAGGAATACTTGCGTCACGGCGAGGAATTTG
ACCTTCAATTCATCTTCCAGCTCTGCAAGATTACTCTCACCGCTGATGTTATGACA
TATATCCATAAGATGGACGCTACCATCCTGGAGGATTGGCAATTTGGACTGACTC
CCCCACCCTCAGCTTCGTTGGAAGACACCTACCGCTTCGTCACAAGTACTGCCA
TTACTTGTCAGAAGAACACTCCACCCAAGGGTAAGGAGGACCCACTTAAGGAG
TACATGTTTTGGGAAGTGGATCTCAAAGAGAAGTTCAGCGCCGACCTGGATCAA
TTTCCTCTGGGTCGTAAGTTCCTCTTGCAAGCAGGACTGCAAGCGGGTCCTGGC
TTGTCGGGTCCTGCCTCGAGCGCCCCTAGAACGTCGACGGGTGGCTCGGCCGTG
GGTAGCTAA
SEQ ID NO. 52: 52L1ΔN13CS3nt
ATGCCTCCAGTACCTGTTTCTAAAGTGGTCTCCACTGATGAATACGTCTCACGTA
CCTCGATTTACTATTACGCTGGTAGTTCAAGACTGTTGACAGTCGGCCACCCATA
CTTTTCTATCAAGAATACGTCCTCAGGAAACGGTAAGAAGGTCCTTGTGCCGAA
AGTTTCGGGTCTCCAATACCGCGTCTTCCGTATCAAGCTGCCTGACCCCAACAAA
TTCGGCTTCCCAGATACTAGTTTCTATAACCCAGAGACCCAGAGACTGGTGTGG
GCCTGCACAGGACTCGAAATTGGCAGGGGTCAACCTTTGGGCGTGGGAATCAG
CGGTCACCCCCTTCTCAATAAGTTCGACGACACAGAGACTTCTAACAAATACGC
TGGTAAGCCAGGCATCGACAACCGTGAATGCCTCTCCATGGATTACAAACAGAC
CCAACTGTGTATTCTGGGATGCAAGCCGCCTATCGGTGAGCATTGGGGTAAAGG
CACACCTTGCAACAATAACTCAGGAAACCCAGGAGACTGCCCACCTTTGCAGCT
TATCAACTCGGTTATTCAAGATGGTGACATGGTCGACACTGGCTTTGGATGTATG
GACTTCAATACTCTCCAGGCTTCCAAGAGCGATGTCCCCATCGACATCTGCTCTT
CCGTGTGTAAATACCCAGATTATCTGCAAATGGCTTCAGAACCTTACGGAGACTC
TCTGTTCTTCTTCTTGCGCAGGGAGCAGATGTTCGTTCGTCACTTTTTCAACAGA
GCCGGTACCTTGGGCGATCCTGTCCCCGGAGACCTTTATATTCAAGGTTCCAACA
GCGGTAACACAGCCACCGTGCAGTCTTCCGCTTTCTTCCCAACTCCTTCAGGCA
GCATGGTGACCAGTGAAAGCCAACTCTTTAATAAGCCTTACTGGTTGCAGAGGG
CTCAAGGACACAACAATGGCATCTGCTGGGGTAACCAGCTGTTCGTTACAGTCG
TCGATACCACTCGTTCTACCAATATGACACTGTGCGCCGAGGTGAAGAAGGAAT
CCACATACAAAAACGAGAATTTCAAGGAATACTTGCGTCACGGCGAGGAATTTG
ACCTTCAATTCATCTTCCAGCTCTGCAAGATTACTCTCACCGCTGATGTTATGACA
TATATCCATAAGATGGACGCTACCATCCTGGAGGATTGGCAATTTGGACTGACTC
CCCCACCCTCAGCTTCGTTGGAAGACACCTACCGCTTCGTCACAAGTACTGCCA
TTACTTGTCAGAAGAACACTCCACCCAAGGGTAAGGAGGACCCACTTAAGGAG
TACATGTTTTGGGAAGTGGATCTCAAAGAGAAGTTCAGCGCCGACCTGGATCAA
TTTCCTCTGGGTCGTAAGTTCCTCTTGCAAGCAGGACTGCAAGCTAGACCTAAA
CTGGCCGGTCCTGCCTCGAGCGCCCCTGCCACGTCGACGGCTGCGGGAGGCGT
GGGTAGCTAA
SEQ ID NO. 53: 52L1NS1ΔC19nt
ATGCCTAGCGAGGCTACCCCTCCAGTACCTGTTTCTAAAGTGGTCTCCACTGATG
AATACGTCTCACGTACCTCGATTTACTATTACGCTGGTAGTTCAAGACTGTTGAC
AGTCGGCCACCCATACTTTTCTATCAAGAATACGTCCTCAGGAAACGGTAAGAA
GGTCCTTGTGCCGAAAGTTTCGGGTCTCCAATACCGCGTCTTCCGTATCAAGCTG
CCTGACCCCAACAAATTCGGCTTCCCAGATACTAGTTTCTATAACCCAGAGACCC
AGAGACTGGTGTGGGCCTGCACAGGACTCGAAATTGGCAGGGGTCAACCTTTG
GGCGTGGGAATCAGCGGTCACCCCCTTCTCAATAAGTTCGACGACACAGAGACT
TCTAACAAATACGCTGGTAAGCCAGGCATCGACAACCGTGAATGCCTCTCCATG
GATTACAAACAGACCCAACTGTGTATTCTGGGATGCAAGCCGCCTATCGGTGAG
CATTGGGGTAAAGGCACACCTTGCAACAATAACTCAGGAAACCCAGGAGACTG
CCCACCTTTGCAGCTTATCAACTCGGTTATTCAAGATGGTGACATGGTCGACACT
GGCTTTGGATGTATGGACTTCAATACTCTCCAGGCTTCCAAGAGCGATGTCCCCA
TCGACATCTGCTCTTCCGTGTGTAAATACCCAGATTATCTGCAAATGGCTTCAGA
ACCTTACGGAGACTCTCTGTTCTTCTTCTTGCGCAGGGAGCAGATGTTCGTTCGT
CACTTTTTCAACAGAGCCGGTACCTTGGGCGATCCTGTCCCCGGAGACCTTTATA
TTCAAGGTTCCAACAGCGGTAACACAGCCACCGTGCAGTCTTCCGCTTTCTTCC
CAACTCCTTCAGGCAGCATGGTGACCAGTGAAAGCCAACTCTTTAATAAGCCTT
ACTGGTTGCAGAGGGCTCAAGGACACAACAATGGCATCTGCTGGGGTAACCAG
CTGTTCGTTACAGTCGTCGATACCACTCGTTCTACCAATATGACACTGTGCGCCG
AGGTGAAGAAGGAATCCACATACAAAAACGAGAATTTCAAGGAATACTTGCGTC
ACGGCGAGGAATTTGACCTTCAATTCATCTTCCAGCTCTGCAAGATTACTCTCAC
CGCTGATGTTATGACATATATCCATAAGATGGACGCTACCATCCTGGAGGATTGGC
AATTTGGACTGACTCCCCCACCCTCAGCTTCGTTGGAAGACACCTACCGCTTCG
TCACAAGTACTGCCATTACTTGTCAGAAGAACACTCCACCCAAGGGTAAGGAGG
ACCCACTTAAGGAGTACATGTTTTGGGAAGTGGATCTCAAAGAGAAGTTCAGCG
CCGACCTGGATCAATTTCCTCTGGGTCGTAAGTTCCTCTTGCAAGCAGGACTGC
AAGCTAGACCTAAACTGTAA
SEQ ID NO. 54: 52L1INS1ΔC25
ATGCCTAGCGAGGCTACCCCTCCAGTACCTGTTTCTAAAGTGGTCTCCACTGATG
AATACGTCTCACGTACCTCGATTTACTATTACGCTGGTAGTTCAAGACTGTTGAC
AGTCGGCCACCCATACTTTTCTATCAAGAATACGTCCTCAGGAAACGGTAAGAA
GGTCCTTGTGCCGAAAGTTTCGGGTCTCCAATACCGCGTCTTCCGTATCAAGCTG
CCTGACCCCAACAAATTCGGCTTCCCAGATACTAGTTTCTATAACCCAGAGACCC
AGAGACTGGTGTGGGCCTGCACAGGACTCGAAATTGGCAGGGGTCAACCTTTG
GGCGTGGGAATCAGCGGTCACCCCCTTCTCAATAAGTTCGACGACACAGAGACT
TCTAACAAATACGCTGGTAAGCCAGGCATCGACAACCGTGAATGCCTCTCCATG
GATTACAAACAGACCCAACTGTGTATTCTGGGATGCAAGCCGCCTATCGGTGAG
CATTGGGGTAAAGGCACACCTTGCAACAATAACTCAGGAAACCCAGGAGACTG
CCCACCTTTGCAGCTTATCAACTCGGTTATTCAAGATGGTGACATGGTCGACACT
GGCTTTGGATGTATGGACTTCAATACTCTCCAGGCTTCCAAGAGCGATGTCCCCA
TCGACATCTGCTCTTCCGTGTGTAAATACCCAGATTATCTGCAAATGGCTTCAGA
ACCTTACGGAGACTCTCTGTTCTTCTTCTTGCGCAGGGAGCAGATGTTCGTTCGT
CACTTTTTCAACAGAGCCGGTACCTTGGGCGATCCTGTCCCCGGAGACCTTTATA
TTCAAGGTTCCAACAGCGGTAACACAGCCACCGTGCAGTCTTCCGCTTTCTTCC
CAACTCCTTCAGGCAGCATGGTGACCAGTGAAAGCCAACTCTTTAATAAGCCTT
ACTGGTTGCAGAGGGCTCAAGGACACAACAATGGCATCTGCTGGGGTAACCAG
CTGTTCGTTACAGTCGTCGATACCACTCGTTCTACCAATATGACACTGTGCGCCG
AGGTGAAGAAGGAATCCACATACAAAAACGAGAATTTCAAGGAATACTTGCGTC
ACGGCGAGGAATTTGACCTTCAATTCATCTTCCAGCTCTGCAAGATTACTCTCAC
CGCTGATGTTATGACATATATCCATAAGATGGACGCTACCATCCTGGAGGATTGGC
AATTTGGACTGACTCCCCCACCCTCAGCTTCGTTGGAAGACACCTACCGCTTCG
TCACAAGTACTGCCATTACTTGTCAGAAGAACACTCCACCCAAGGGTAAGGAGG
ACCCACTTAAGGAGTACATGTTTTGGGAAGTGGATCTCAAAGAGAAGTTCAGCG
CCGACCTGGATCAATTTCCTCTGGGTCGTAAGTTCCTCTTGCAAGCAGGACTGTA
A
SEQ ID NO. 55: 52L1NS2ΔC19nt
ATGTCCGAGCGTCCTCCAGTACCTGTTTCTAAAGTGGTCTCCACTGATGAATACG
TCTCACGTACCTCGATTTACTATTACGCTGGTAGTTCAAGACTGTTGACAGTCGG
CCACCCATACTTTTCTATCAAGAATACGTCCTCAGGAAACGGTAAGAAGGTCCTT
GTGCCGAAAGTTTCGGGTCTCCAATACCGCGTCTTCCGTATCAAGCTGCCTGACC
CCAACAAATTCGGCTTCCCAGATACTAGTTTCTATAACCCAGAGACCCAGAGACT
GGTGTGGGCCTGCACAGGACTCGAAATTGGCAGGGGTCAACCTTTGGGCGTGG
GAATCAGCGGTCACCCCCTTCTCAATAAGTTCGACGACACAGAGACTTCTAACA
AATACGCTGGTAAGCCAGGCATCGACAACCGTGAATGCCTCTCCATGGATTACA
AACAGACCCAACTGTGTATTCTGGGATGCAAGCCGCCTATCGGTGAGCATTGGG
GTAAAGGCACACCTTGCAACAATAACTCAGGAAACCCAGGAGACTGCCCACCT
TTGCAGCTTATCAACTCGGTTATTCAAGATGGTGACATGGTCGACACTGGCTTTG
GATGTATGGACTTCAATACTCTCCAGGCTTCCAAGAGCGATGTCCCCATCGACAT
CTGCTCTTCCGTGTGTAAATACCCAGATTATCTGCAAATGGCTTCAGAACCTTAC
GGAGACTCTCTGTTCTTCTTCTTGCGCAGGGAGCAGATGTTCGTTCGTCACTTTT
TCAACAGAGCCGGTACCTTGGGCGATCCTGTCCCCGGAGACCTTTATATTCAAGG
TTCCAACAGCGGTAACACAGCCACCGTGCAGTCTTCCGCTTTCTTCCCAACTCC
TTCAGGCAGCATGGTGACCAGTGAAAGCCAACTCTTTAATAAGCCTTACTGGTT
GCAGAGGGCTCAAGGACACAACAATGGCATCTGCTGGGGTAACCAGCTGTTCG
TTACAGTCGTCGATACCACTCGTTCTACCAATATGACACTGTGCGCCGAGGTGAA
GAAGGAATCCACATACAAAAACGAGAATTTCAAGGAATACTTGCGTCACGGCGA
GGAATTTGACCTTCAATTCATCTTCCAGCTCTGCAAGATTACTCTCACCGCTGAT
GTTATGACATATATCCATAAGATGGACGCTACCATCCTGGAGGATTGGCAATTTGG
ACTGACTCCCCCACCCTCAGCTTCGTTGGAAGACACCTACCGCTTCGTCACAAG
TACTGCCATTACTTGTCAGAAGAACACTCCACCCAAGGGTAAGGAGGACCCACT
TAAGGAGTACATGTTTTGGGAAGTGGATCTCAAAGAGAAGTTCAGCGCCGACCT
GGATCAATTTCCTCTGGGTCGTAAGTTCCTCTTGCAAGCAGGACTGCAAGCTAG
ACCTAAACTGTAA
SEQ ID NO. 56: 52L1NS3ΔC19nt
ATGTCCGAGCCTCCAGTACCTGTTTCTAAAGTGGTCTCCACTGATGAATACGTCT
CACGTACCTCGATTTACTATTACGCTGGTAGTTCAAGACTGTTGACAGTCGGCCA
CCCATACTTTTCTATCAAGAATACGTCCTCAGGAAACGGTAAGAAGGTCCTTGTG
CCGAAAGTTTCGGGTCTCCAATACCGCGTCTTCCGTATCAAGCTGCCTGACCCCA
ACAAATTCGGCTTCCCAGATACTAGTTTCTATAACCCAGAGACCCAGAGACTGGT
GTGGGCCTGCACAGGACTCGAAATTGGCAGGGGTCAACCTTTGGGCGTGGGAA
TCAGCGGTCACCCCCTTCTCAATAAGTTCGACGACACAGAGACTTCTAACAAAT
ACGCTGGTAAGCCAGGCATCGACAACCGTGAATGCCTCTCCATGGATTACAAAC
AGACCCAACTGTGTATTCTGGGATGCAAGCCGCCTATCGGTGAGCATTGGGGTA
AAGGCACACCTTGCAACAATAACTCAGGAAACCCAGGAGACTGCCCACCTTTG
CAGCTTATCAACTCGGTTATTCAAGATGGTGACATGGTCGACACTGGCTTTGGAT
GTATGGACTTCAATACTCTCCAGGCTTCCAAGAGCGATGTCCCCATCGACATCTG
CTCTTCCGTGTGTAAATACCCAGATTATCTGCAAATGGCTTCAGAACCTTACGGA
GACTCTCTGTTCTTCTTCTTGCGCAGGGAGCAGATGTTCGTTCGTCACTTTTTCA
ACAGAGCCGGTACCTTGGGCGATCCTGTCCCCGGAGACCTTTATATTCAAGGTTC
CAACAGCGGTAACACAGCCACCGTGCAGTCTTCCGCTTTCTTCCCAACTCCTTC
AGGCAGCATGGTGACCAGTGAAAGCCAACTCTTTAATAAGCCTTACTGGTTGCA
GAGGGCTCAAGGACACAACAATGGCATCTGCTGGGGTAACCAGCTGTTCGTTAC
AGTCGTCGATACCACTCGTTCTACCAATATGACACTGTGCGCCGAGGTGAAGAA
GGAATCCACATACAAAAACGAGAATTTCAAGGAATACTTGCGTCACGGCGAGGA
ATTTGACCTTCAATTCATCTTCCAGCTCTGCAAGATTACTCTCACCGCTGATGTTA
TGACATATATCCATAAGATGGACGCTACCATCCTGGAGGATTGGCAATTTGGACT
GACTCCCCCACCCTCAGCTTCGTTGGAAGACACCTACCGCTTCGTCACAAGTAC
TGCCATTACTTGTCAGAAGAACACTCCACCCAAGGGTAAGGAGGACCCACTTAA
GGAGTACATGTTTTGGGAAGTGGATCTCAAAGAGAAGTTCAGCGCCGACCTGGA
TCAATTTCCTCTGGGTCGTAAGTTCCTCTTGCAAGCAGGACTGCAAGCTAGACCT
AAACTGTAA
SEQ ID NO. 57: 52L1NS4ΔC19nt
ATGTCCCCTCCAGTACCTGTTTCTAAAGTGGTCTCCACTGATGAATACGTCTCAC
GTACCTCGATTTACTATTACGCTGGTAGTTCAAGACTGTTGACAGTCGGCCACCC
ATACTTTTCTATCAAGAATACGTCCTCAGGAAACGGTAAGAAGGTCCTTGTGCCG
AAAGTTTCGGGTCTCCAATACCGCGTCTTCCGTATCAAGCTGCCTGACCCCAAC
AAATTCGGCTTCCCAGATACTAGTTTCTATAACCCAGAGACCCAGAGACTGGTGT
GGGCCTGCACAGGACTCGAAATTGGCAGGGGTCAACCTTTGGGCGTGGGAATC
AGCGGTCACCCCCTTCTCAATAAGTTCGACGACACAGAGACTTCTAACAAATAC
GCTGGTAAGCCAGGCATCGACAACCGTGAATGCCTCTCCATGGATTACAAACAG
ACCCAACTGTGTATTCTGGGATGCAAGCCGCCTATCGGTGAGCATTGGGGTAAA
GGCACACCTTGCAACAATAACTCAGGAAACCCAGGAGACTGCCCACCTTTGCA
GCTTATCAACTCGGTTATTCAAGATGGTGACATGGTCGACACTGGCTTTGGATGT
ATGGACTTCAATACTCTCCAGGCTTCCAAGAGCGATGTCCCCATCGACATCTGCT
CTTCCGTGTGTAAATACCCAGATTATCTGCAAATGGCTTCAGAACCTTACGGAGA
CTCTCTGTTCTTCTTCTTGCGCAGGGAGCAGATGTTCGTTCGTCACTTTTTCAAC
AGAGCCGGTACCTTGGGCGATCCTGTCCCCGGAGACCTTTATATTCAAGGTTCCA
ACAGCGGTAACACAGCCACCGTGCAGTCTTCCGCTTTCTTCCCAACTCCTTCAG
GCAGCATGGTGACCAGTGAAAGCCAACTCTTTAATAAGCCTTACTGGTTGCAGA
GGGCTCAAGGACACAACAATGGCATCTGCTGGGGTAACCAGCTGTTCGTTACAG
TCGTCGATACCACTCGTTCTACCAATATGACACTGTGCGCCGAGGTGAAGAAGG
AATCCACATACAAAAACGAGAATTTCAAGGAATACTTGCGTCACGGCGAGGAAT
TTGACCTTCAATTCATCTTCCAGCTCTGCAAGATTACTCTCACCGCTGATGTTATG
ACATATATCCATAAGATGGACGCTACCATCCTGGAGGATTGGCAATTTGGACTGA
CTCCCCCACCCTCAGCTTCGTTGGAAGACACCTACCGCTTCGTCACAAGTACTG
CCATTACTTGTCAGAAGAACACTCCACCCAAGGGTAAGGAGGACCCACTTAAGG
AGTACATGTTTTGGGAAGTGGATCTCAAAGAGAAGTTCAGCGCCGACCTGGATC
AATTTCCTCTGGGTCGTAAGTTCCTCTTGCAAGCAGGACTGCAAGCTAGACCTA
AACTGTAA
SEQ ID NO. 58: 52L1ΔN14ΔC25nt
ATGCCAGTACCTGTTTCTAAAGTGGTCTCCACTGATGAATACGTCTCACGTACCT
CGATTTACTATTACGCTGGTAGTTCAAGACTGTTGACAGTCGGCCACCCATACTT
TTCTATCAAGAATACGTCCTCAGGAAACGGTAAGAAGGTCCTTGTGCCGAAAGT
TTCGGGTCTCCAATACCGCGTCTTCCGTATCAAGCTGCCTGACCCCAACAAATTC
GGCTTCCCAGATACTAGTTTCTATAACCCAGAGACCCAGAGACTGGTGTGGGCC
TGCACAGGACTCGAAATTGGCAGGGGTCAACCTTTGGGCGTGGGAATCAGCGG
TCACCCCCTTCTCAATAAGTTCGACGACACAGAGACTTCTAACAAATACGCTGGT
AAGCCAGGCATCGACAACCGTGAATGCCTCTCCATGGATTACAAACAGACCCAA
CTGTGTATTCTGGGATGCAAGCCGCCTATCGGTGAGCATTGGGGTAAAGGCACA
CCTTGCAACAATAACTCAGGAAACCCAGGAGACTGCCCACCTTTGCAGCTTATC
AACTCGGTTATTCAAGATGGTGACATGGTCGACACTGGCTTTGGATGTATGGACT
TCAATACTCTCCAGGCTTCCAAGAGCGATGTCCCCATCGACATCTGCTCTTCCGT
GTGTAAATACCCAGATTATCTGCAAATGGCTTCAGAACCTTACGGAGACTCTCTG
TTCTTCTTCTTGCGCAGGGAGCAGATGTTCGTTCGTCACTTTTTCAACAGAGCCG
GTACCTTGGGCGATCCTGTCCCCGGAGACCTTTATATTCAAGGTTCCAACAGCGG
TAACACAGCCACCGTGCAGTCTTCCGCTTTCTTCCCAACTCCTTCAGGCAGCATG
GTGACCAGTGAAAGCCAACTCTTTAATAAGCCTTACTGGTTGCAGAGGGCTCAA
GGACACAACAATGGCATCTGCTGGGGTAACCAGCTGTTCGTTACAGTCGTCGAT
ACCACTCGTTCTACCAATATGACACTGTGCGCCGAGGTGAAGAAGGAATCCACA
TACAAAAACGAGAATTTCAAGGAATACTTGCGTCACGGCGAGGAATTTGACCTT
CAATTCATCTTCCAGCTCTGCAAGATTACTCTCACCGCTGATGTTATGACATATAT
CCATAAGATGGACGCTACCATCCTGGAGGATTGGCAATTTGGACTGACTCCCCCA
CCCTCAGCTTCGTTGGAAGACACCTACCGCTTCGTCACAAGTACTGCCATTACTT
GTCAGAAGAACACTCCACCCAAGGGTAAGGAGGACCCACTTAAGGAGTACATG
TTTTGGGAAGTGGATCTCAAAGAGAAGTTCAGCGCCGACCTGGATCAATTTCCT
CTGGGTCGTAAGTTCCTCTTGCAAGCAGGACTGTAA.

1. A modified HPV52 L1 protein comprising a modification selected from the group consisting of the following or a combination thereof, when compared with a wild-type HPV52 L1 protein:
mutating the amino acid at position 447 from aspartate to glutamate;
deleting 1 to 20 successive or nonsuccessive amino acids at the N-terminus;
deleting 1 to 25 successive or nonsuccessive amino acids at the C-terminus;
substituting one or more amino acids at positions 1 to 20 at the N-terminus; and
substituting one or more amino acids at positions 1 to 25 at the C-terminus.

2. The modified HPV52 L1 protein according to claim 1, which is as shown in the sequence selected from SEQ ID No. 2 to SEQ ID No. 29.

3. A polynucleotide encoding the modified HPV52 L1 protein according to claim 1,
wherein the sequence of the polynucleotide is whole-gene optimized using insect cell codons.

4. The polynucleotide according to claim 3, which is as shown in the sequence selected from SEQ ID No. 31 to SEQ ID No. 58.

5. A vector comprising the polynucleotide according to claim 3,
wherein the vector is selected from the group consisting of plasmid, recombinant Bacmid, and recombinant baculovirus.

6. A host cell comprising the vector according to claim 5,
wherein the host cell is selected from the group consisting of E. coli, yeast cell and insect cell.

7. A multimer wherein:
the multimer is a pentamer or a virus-like particle;
the multimer comprises the modified HPV52 L1 protein according to claim 1, or is formed by the modified HPV52 L1 protein according to claim 1.

8. A vaccine for the prevention of papillomavirus infection or related diseases, said vaccine comprises:
the multimer according to claim 7,
an adjuvant, and
an excipient or carrier for vaccines;
wherein the adjuvant is an adjuvant for human use.

9. (canceled)

10. (canceled)

11. The modified HPV52 L1 protein according to claim 1, wherein the wild-type HPV52 L1 protein is as shown in the sequence selected from the group consisting of NCBI Accession No. AEI61557.1, ABU55797.1, AEI61589.1, AIF71344.1, APQ44868.1, AEI61581.1, AIF71350.1, and CAD1814034.1.

12. The modified HPV52 L1 protein according to claim 1, wherein the wild-type HPV52 L1 protein is as shown in SEQ ID No. 1.

13. The modified HPV52 L1 protein according to claim 1, deleting 2, 4, 5, 8, 10, 13, 14, 15, 18, or 20 successive or nonsuccessive amino acids at the N-terminus.

14. The modified HPV52 L1 protein according to claim 1, deleting 13 amino acids at the N-terminus and substituting with any one selected from the group consisting of serine, serine-glutamate, serine-glutamate-arginine, and proline-serine-glutamate-alanine-threonine.

15. The modified HPV52 L1 protein according to claim 1, deleting 19 or 25 successive or nonsuccessive amino acids at the C-terminus.

16. The modified HPV52 L1 protein according to claim 1, substituting one or more basic amino acids at positions 1 to 23 at the C-terminus with any one selected from the group consisting of polar uncharged amino acid, non-polar amino acid, and acidic amino acid;
wherein the basic amino acid is selected from arginine and/or lysine;
wherein the polar uncharged amino acid is selected from the group consisting of glycine, serine, and threonine;
wherein the non-polar amino acid is selected from alanine and/or valine; and
wherein the acidic amino acid is selected from aspartate and/or glutamate.

17. The vaccine according to claim 8, further comprising one of the following or a combination thereof: mucosa-tropic HPV virus-like particle or chimeric virus-like particle, skin-tropic HPV virus-like particle or chimeric virus-like particle.


SEQUENCE LISTING (58 sequences)

SEQ ID NO: 1 - protein, 503 aa, Human papillomavirus type 52
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 2 - protein, 484 aa, Artificial Sequence
  52L1D447E delta C19
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 3 - protein, 483 aa, Artificial Sequence
  52L1 delta N2
MVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 4 - protein, 481 aa, Artificial Sequence
  52L1 delta N4
MRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 5 - protein, 480 aa, Artificial Sequence
  52L1 delta N5
MPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 6 - protein, 477 aa, Artificial Sequence
  52L1 delta N8
MATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 7 - protein, 475 aa, Artificial Sequence
  52L1 delta N10
MVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 8 - protein, 472 aa, Artificial Sequence
  52L1 delta N13
MPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 9 - protein, 470 aa, Artificial Sequence
  52L1 delta N15
MVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 10 - protein, 467 aa, Artificial Sequence
  52L1 delta N18
MSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 11 - protein, 465 aa, Artificial Sequence
  52L1 delta N20
MVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 12 - protein, 503 aa, Artificial Sequence
  52L1CS1
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPGLKGPASSAPRTSTDGSGVGR

SEQ ID NO: 13 - protein, 503 aa, Artificial Sequence
  52L1CS2
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPGLKGPASSAPRTSTDGSGVDG

SEQ ID NO: 14 - protein, 503 aa, Artificial Sequence
  52L1CS3
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPGLGSPASSAPRTSTDGSGVKR

SEQ ID NO: 15 - protein, 503 aa, Artificial Sequence
  52L1CS4
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPGLGSPASSAPRTSTDGSGVDR

SEQ ID NO: 16 - protein, 503 aa, Artificial Sequence
  52L1CS5
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKLAGPASSAPATSTAAGGVGS

SEQ ID NO: 17 - protein, 503 aa, Artificial Sequence
  52L1CS6
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKLEAPASSAPGTSTGSKAVAG

SEQ ID NO: 18 - protein, 503 aa, Artificial Sequence
  52L1CS7
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKLAGPASSAPATSTDGSGVKR

SEQ ID NO: 19 - protein, 503 aa, Artificial Sequence
  52L1CS8
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKLAGPASSAPRTSTDGSGVKR

SEQ ID NO: 20 - protein, 503 aa, Artificial Sequence
  52L1CS9
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQAGPGLSGPASSAPRTSTGGSAVGS

SEQ ID NO: 21 - protein, 491 aa, Artificial Sequence
  52L1 delta N13CS1
MPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPGLSGPASSAPRTSTGGSAVGS

SEQ ID NO: 22 - protein, 491 aa, Artificial Sequence
  52L1 delta N13CS2
MPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQAGPGLSGPASSAPRTSTGGSAVGS

SEQ ID NO: 23 - protein, 491 aa, Artificial Sequence
  52L1 delta N13CS3
MPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKLAGPASSAPATSTAAGGVGS

SEQ ID NO: 24 - protein, 477 aa, Artificial Sequence
  52L1NS1 delta C19
MPSEATPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 25 - protein, 471 aa, Artificial Sequence
  52L1NS1 delta C25
MPSEATPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGL

SEQ ID NO: 26 - protein, 475 aa, Artificial Sequence
  52L1NS2 delta C19
MSERPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 27 - protein, 474 aa, Artificial Sequence
  52L1NS3 delta C19
MSEPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 28 - protein, 473 aa, Artificial Sequence
  52L1NS4 delta C19
MSPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGLQARPKL

SEQ ID NO: 29 - protein, 465 aa, Artificial Sequence
  52L1 delta N14 delta C25
MPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKEYMFWEVDLKEKFSADLDQFPLGRKFLLQAGL

SEQ ID NO: 30 - DNA, 1512 bp, Human papillomavirus type 52
atgtccgtgtggcggcctagtgaggccactgtgtacctgcctcctgtacctgtctctaaggttgtaagcactgatgagtatgtgtctcgcacaagcatctattattatgcaggcagttctcgattactaacagtaggacatccctatttttctattaaaaacaccagtagtggtaatggtaaaaaagttttagttcccaaggtgtctggcctgcaatacagggtatttagaattaaattgccggaccctaataaatttggttttccggatacatctttttataacccagaaacccaaaggttggtgtgggcctgtacaggcttggaaattggtaggggacagcctttaggtgtgggtattagtgggcatcctttattaaacaagtttgatgatactgaaaccagtaacaaatatgctggtaaacctggtatagataatagagaatgtttatctatggattataagcagactcagttatgcattttaggatgcaaacctcctataggtgaacattggggtaagggaaccccttgtaataataattcaggaaatcctggggattgtcctcccctacaactcattaacagtgtaatacaggatggggacatggtagatacaggatttggttgcatggattttaataccttgcaagctagtaaaagtgatgtgcccattgatatatgtagcagtgtatgtaagtatccagattatttgcaaatggctagcgagccatatggtgacagtttgttcttttttcttagacgtgagcaaatgtttgttagacacttttttaatagggctggtaccttaggtgaccctgtgccaggtgatttatatatacaagggtctaactctggcaatactgccactgtacaaagcagtgctttttttcctactcctagtggttctatggtaacctcagaatcccaattatttaataaaccgtactggttacaacgtgcgcagggccacaataatggcatatgttggggcaatcagttgtttgtcacagttgtggataccactcgtagcactaacatgactttatgtgctgaagttaaaaaggaaagcacatataaaaatgaaaattttaaggaataccttcgtcatggcgaggaatttgatttacaatttatttttcaattgtgcaaaattacattaacagctgatgttatgacatacattcataagatggatgccactattttagaggactggcaatttggccttaccccaccaccgtctgcatctttggaggacacatacagatttgtaacttctactgctataacttgtcaaaaaaacacaccacctaaaggaaaggaagatcctttaaaggactatatgttttgggaggtggatttaaaagaaaagttttctgcagatttagatcagtttcctttaggtaggaagtttttgttacaggcagggctacaggctaggcccaaactaaaacgccctgcatcatcagccccacgtacctccacaaagaagaaaaaggttaaaaggtaa

SEQ ID NO: 31 - DNA, 1455 bp, Artificial Sequence
  52L1D447E delta C19nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 32 - DNA, 1452 bp, Artificial Sequence
  52L1 delta N2nt
atggtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 33 - DNA, 1446 bp, Artificial Sequence
  52L1 delta N4nt
atgcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 34 - DNA, 1443 bp, Artificial Sequence
  52L1 delta N5nt
atgccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 35 - DNA, 1434 bp, Artificial Sequence
  52L1 delta N8nt
atggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 36 - DNA, 1428 bp, Artificial Sequence
  52L1 delta N10nt
atggtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 37 - DNA, 1419 bp, Artificial Sequence
  52L1 delta N13nt
atgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 38 - DNA, 1413 bp, Artificial Sequence
  52L1 delta N15nt
atggtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 39 - DNA, 1404 bp, Artificial Sequence
  52L1 delta N18nt
atgtctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 40 - DNA, 1398 bp, Artificial Sequence
  52L1 delta N20nt
atggtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 41 - DNA, 1512 bp, Artificial Sequence
  52L1CS1nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctcgtcctggactgaaaggtcctgcatcgagcgctcctagaacgtcgacggacggctcgggagtgggacgctaa

SEQ ID NO: 42 - DNA, 1512 bp, Artificial Sequence
  52L1CS2nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctcgtcctggactgaaaggtcctgcatcgagcgctcctagaacgtcgacggacggctcgggagtggacggctaa

SEQ ID NO: 43 - DNA, 1512 bp, Artificial Sequence
  52L1CS3nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctcgtcctggactgggatcgcctgcatcgagcgctcctagaacgtcgacggacggctcgggagtgaaacgctaa

SEQ ID NO: 44 - DNA, 1512 bp, Artificial Sequence
  52L1CS4nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctcgtcctggactgggatcgcctgcatcgagcgctcctagaacgtcgacggacggctcgggagtggaccgctaa

SEQ ID NO: 45 - DNA, 1512 bp, Artificial Sequence
  52L1CS5nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactggctggtcctgcctcttccgcacccgcgacttcaaccgctgccggcggagttgggtcgtaa

SEQ ID NO: 46 - DNA, 1512 bp, Artificial Sequence
  52L1CS6nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactggaagctcctgcctcttccgcacccggtacttcaaccggctcgaaagcggttgctggataa

SEQ ID NO: 47 - DNA, 1512 bp, Artificial Sequence
  52L1CS7nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactggctggtcctgcttcctcagctccagctacctcaaccgacggttctggtgtgaagcgctaa

SEQ ID NO: 48 - DNA, 1512 bp, Artificial Sequence
  52L1CS8nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactggctggtcctgcttcctcagctccacgtacctcaaccgacggttctggtgtgaagcgctaa

SEQ ID NO: 49 - DNA, 1512 bp, Artificial Sequence
  52L1CS9nt
atgtccgtgtggcgtccttccgaggctactgtgtacttgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagcgggtcctggcttgtcgggtcctgcctcgagcgcccctagaacgtcgacgggtggctcggccgtgggtagctaa

SEQ ID NO: 50 - DNA, 1476 bp, Artificial Sequence
  52L1 delta N13CS1nt
atgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagcgagacctggcttgtcgggtcctgcctcgagcgcccctagaacgtcgacgggtggctcggccgtgggtagctaa

SEQ ID NO: 51 - DNA, 1476 bp, Artificial Sequence
  52L1 delta N13CS2nt
atgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagcgggtcctggcttgtcgggtcctgcctcgagcgcccctagaacgtcgacgggtggctcggccgtgggtagctaa

SEQ ID NO: 52 - DNA, 1476 bp, Artificial Sequence
  52L1 delta N13CS3nt
atgcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactggccggtcctgcctcgagcgcccctgccacgtcgacggctgcgggaggcgtgggtagctaa

SEQ ID NO: 53 - DNA, 1434 bp, Artificial Sequence
  52L1NS1 delta C19nt
atgcctagcgaggctacccctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 54 - DNA, 1416 bp, Artificial Sequence
  52L1NS1 delta C25
atgcctagcgaggctacccctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgtaa

SEQ ID NO: 55 - DNA, 1428 bp, Artificial Sequence
  52L1NS2 delta C19nt
atgtccgagcgtcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 56 - DNA, 1425 bp, Artificial Sequence
  52L1NS3 delta C19nt
atgtccgagcctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 57 - DNA, 1422 bp, Artificial Sequence
  52L1NS4 delta C19nt
atgtcccctccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgcaagctagacctaaactgtaa

SEQ ID NO: 58 - DNA, 1398 bp, Artificial Sequence
  52L1 delta N14 delta C25nt
atgccagtacctgtttctaaagtggtctccactgatgaatacgtctcacgtacctcgatttactattacgctggtagttcaagactgttgacagtcggccacccatacttttctatcaagaatacgtcctcaggaaacggtaagaaggtccttgtgccgaaagtttcgggtctccaataccgcgtcttccgtatcaagctgcctgaccccaacaaattcggcttcccagatactagtttctataacccagagacccagagactggtgtgggcctgcacaggactcgaaattggcaggggtcaacctttgggcgtgggaatcagcggtcacccccttctcaataagttcgacgacacagagacttctaacaaatacgctggtaagccaggcatcgacaaccgtgaatgcctctccatggattacaaacagacccaactgtgtattctgggatgcaagccgcctatcggtgagcattggggtaaaggcacaccttgcaacaataactcaggaaacccaggagactgcccacctttgcagcttatcaactcggttattcaagatggtgacatggtcgacactggctttggatgtatggacttcaatactctccaggcttccaagagcgatgtccccatcgacatctgctcttccgtgtgtaaatacccagattatctgcaaatggcttcagaaccttacggagactctctgttcttcttcttgcgcagggagcagatgttcgttcgtcactttttcaacagagccggtaccttgggcgatcctgtccccggagacctttatattcaaggttccaacagcggtaacacagccaccgtgcagtcttccgctttcttcccaactccttcaggcagcatggtgaccagtgaaagccaactctttaataagccttactggttgcagagggctcaaggacacaacaatggcatctgctggggtaaccagctgttcgttacagtcgtcgataccactcgttctaccaatatgacactgtgcgccgaggtgaagaaggaatccacatacaaaaacgagaatttcaaggaatacttgcgtcacggcgaggaatttgaccttcaattcatcttccagctctgcaagattactctcaccgctgatgttatgacatatatccataagatggacgctaccatcctggaggattggcaatttggactgactcccccaccctcagcttcgttggaagacacctaccgcttcgtcacaagtactgccattacttgtcagaagaacactccacccaagggtaaggaggacccacttaaggagtacatgttttgggaagtggatctcaaagagaagttcagcgccgacctggatcaatttcctctgggtcgtaagttcctcttgcaagcaggactgtaa